(19) United States
(12) Patent Application Publication
(10) Pub. No.: US 2021/0317187 A1
(43) Pub. Date: Oct. 14, 2021

(54) INTRACELLULAR DELIVERY OF BIOMOLECULES TO MODULATE ANTIBODY PRODUCTION

(71) Applicant: SQZ Biotechnologies Company, Watertown, MA (US)

(72) Inventors: Armon R. SHAREI, Watertown, MA (US); Jonathan B. GILBERT, Somerville, MA (US)

(21) Appl. No.: 16/769,993

(22) PCT Filed: Dec. 4, 2018
(86) PCT No.: PCT/US2018/063931

§ 371 Date: Jun. 4, 2020

Related U.S. Application Data
(60) Provisional application No. 62/594,981, filed on Dec. 5, 2017.

(51) Int. Cl. C07K 16/00; C12N 5/0781; A61K 35/17; C12N 15/87

(52) U.S. Cl. CPC C07K 16/00; C12N 5/0635; A61K 35/17; C12N 15/87; C12N 2510/00; C07K 2317/14; C07K 2317/21; C07K 2317/24; C12N 2527/00

(57) ABSTRACT

The present invention provides methods and systems for modulating antibody production by passing a cell suspension through a constriction, wherein the constriction deforms the cell thereby causing a perturbation of the cell such that a compound that modulates antibody production enters the cell. In some embodiments, the invention provides methods and systems for inducing de novo antibody production in cells.

### CROSS-REFERENCE TO RELATED APPLICATION

This application claims priority to U.S. Provisional application No. 62/594,981 filed on Dec. 5, 2017, the contents of which are incorporated herein by reference in their entirety.

### FIELD OF THE INVENTION

The present disclosure relates generally to methods for modulating antibody production by delivering a compound into a cell by passing a cell suspension through a cell-deforming constriction.

### BACKGROUND

Antibodies are commonly used for numerous diagnostic, research, and therapeutic purposes and have a critical role in mediating an effective immune response. During the humoral immune response, a complex set of signaling events coordinate antibody production. Sufficient in-vivo antibody production is critical for combating infectious diseases, mediating anti-tumor responses during cancer, and generating effective vaccine-mediated immunity. Conversely, antibody production directed at endogenous antigens contributes to pathogenic autoimmune responses.

Rapid generation of an antibody repertoire with the desired antibody quantity and specificity is a critical part of mounting an effective humoral immune response. Intracellular delivery of proteins, nucleic acids, or small molecules that stimulate endogenous antibody production or induce de novo antibody production can enable fine tuning of antigen-specific antibody production for research and therapeutic purposes. For example, inducing an augmented or de novo humoral immune response directed against tumor-associated antigens may be useful for treating cancer.

Current intracellular delivery methods are not effective at delivering molecules to sensitive cell types, such as primary immune cells and stem cells, in order to stimulate or induce antibody production. Thus, there is an unmet need for intracellular delivery techniques that are highly effective at delivering a range of molecules to modulate antibody production. In addition, methods that allow for rapid, high throughput intracellular delivery of molecules to modulate antibody production can be applied more effectively to large scale clinical and industrial applications utilizing therapeutic and research and development antibodies. References that describe methods of using channels to deliver compounds to cells include WO2013059343, WO2015023982, and PCT/US2015/058489.

All references cited herein, including patent applications and publications, are incorporated by reference in their entirety.

### BRIEF SUMMARY OF THE INVENTION

Certain aspects of the present invention provides a method for altering endogenous antibody production in an antibody-producing cell, the method comprising passing a cell suspension comprising the antibody-producing cell through a constriction, wherein said constriction deforms the cell thereby causing a perturbation of the cell such that a compound that alters antibody production enters the antibody-producing cell, wherein endogenous antibody production in said antibody-producing cell is altered. In some embodiments, the endogenous antibody production is enhanced. In some embodiments, the endogenous antibody production is decreased. In some embodiments, the constriction is contained within a microfluidic channel. In some embodiments, the constriction is a pore or contained within a pore. In some embodiments, the pore is contained in a surface. In some embodiments, the surface is a filter. In some embodiments, the surface is a membrane. In some embodiments, the constriction size is a function of the cell diameter. In some embodiments, the constriction size is about 20%, about 30%, about 40%, about 50%, about 60%, about 70%, about 80%, about 90%, or about 99% of the cell diameter.

In some embodiments that can be combined with the previous embodiments, the cell suspension comprises a mixed cell population. In some embodiments, the cell suspension is whole blood. In some embodiments, the cell suspension comprises a purified cell population. In some embodiments, the cell suspension comprises mammalian cells. In some embodiments, the cell suspension comprises monkey, mouse, dog, cat, horse, rat, sheep, goat, or rabbit cells. In some embodiments, the cell suspension comprises human cells. In some embodiments, the cell suspension comprises non-mammalian cells. In some embodiments, the cell suspension comprises bacteria, yeast, chicken, frog, insect, or nematode cells. In some embodiments, the cell suspension comprises peripheral blood mononuclear cells. In some embodiments, the antibody-producing cell is an immune cell. In some embodiments, the antibody-producing cell is a B cell or B cell precursor. In some embodiments, the antibody-producing cell is a B cell precursor, naïve B cell, activated B cell, memory B cell, plasma cell, B-1 cell, marginal-zone B cell, follicular B cell, regulatory B cell, or B cell lymphoma cell. In some embodiments, the antibody-producing cell is a bone-marrow derived B cell precursor.

In some embodiments that can be combined with the previous embodiments, the compound comprises a nucleic acid. In some embodiments, nucleic acid encodes a siRNA, mRNA, miRNA, lncRNA, tRNA, saRNA or shRNA. In some embodiments, the nucleic acid is a plasmid. In some embodiments, the compound comprises a peptide nucleic acid (PNA). In some embodiments, the compound comprises a protein-nucleic acid complex. In some embodiments, the protein-nucleic acid complex comprises a Cas9 protein and a guide RNA. In some embodiments, the protein-nucleic acid complex further comprises donor DNA. In some embodiments, the nucleic acid encodes a Cas9 protein and a guide RNA. In some embodiments, the protein-nucleic acid complex further comprises donor DNA.

In some embodiments, the compound comprises a protein or polypeptide. In some embodiments, the protein is a TALEN protein, Zinc finger nuclease, mega nuclease, or CRE recombinase. In some embodiments, the protein is a transcription factor. In some embodiments, the protein is a transposase or integrase enzyme. In some embodiments, the protein is an anti-apoptotic protein.

In some embodiments, the compound is B-cell activating factor. In some embodiments, the compound is a proliferation inducing ligand. In some embodiments, the compound is an activator of a B cell receptor signaling molecule. In some embodiments, the compound is ATP. In some embodiments, the compound is a cell activation factor. In some embodiments, the compound is a cell differentiation factor. In some embodiments, the compound is a small molecule. In some embodiments, the compound is in a nanoparticle. In some embodiments, the compound is in a liposome. In some embodiments, the compound is in a nucleic acid delivery vehicle. In some embodiments, the compound is in a virus. In some embodiments, the compound is in a viral particle. In some embodiments, the compound is in a vehicle comprising viral capsid. In some embodiments, the compound is in an adeno-associated virus. In some embodiments, the compound is in an adeno-associated virus particle. In some embodiments, the compound is in a vehicle comprising adeno-associated virus capsid.

In some embodiments, the antibody is a human or humanized antibody. In some embodiments, the antibody is an antigen binding antibody variant. In some embodiments, the antibody class is IgM, IgG, IgA, IgE, or IgD. In some embodiments, the antibody is an antigen binding antibody fragment. In some embodiments, the antibody is a Fab, Fab′, Fab′-SH, Fab₂, F(ab′)₂, Fv, scFv, scFab, or dsFv fragment. In some embodiments, the antibody is a full length antibody. In some embodiments, the antibody is a single-domain antibody. In some embodiments, the antibody is a nanobody, V_{H}H or V_{NAR} antibody fragment. In some embodiments, the antibody is a single-chain antibody. In some embodiments, the antibody is a multi-specific antibody. In some embodiments, the antibody is an antibody fusion protein.

In some embodiments, said cell suspension is contacted with the compound before, concurrently, or after passing through the constriction. In some embodiments, the channel comprises a constriction length of about 30 μm and a constriction width of about 4 μm. In some embodiments, the channel comprises a constriction length of about 10 μm and a constriction width of about 4 μm. In some embodiments, the pore size is about 0.4 μm, about 4 μm, about 5 μm, about 8 μm, about 10 μm, about 12 μm, or about 14 μm. In some embodiments, the method is performed between about −5° C. and about 45° C. In some embodiments, the endogenous antibody production is altered by at least about 25%, about 50%, about 75%, about 100%, about 150%, about 200%, or more than about 200%. In some embodiments, the endogenous antibody production is sustained for about 25%, about 50%, about 75%, about 100%, about 150%, about 200%, or more than about 200% longer than antibody production by cells that did not pass through the constriction.

In some embodiments, a patient is treated by introducing the antibody-producing immune cell modified according to any one of the aforementioned methods to the patient. In some embodiments, the cell is isolated from a patient, modified according to any one of the aforementioned methods, and introduced back into the patient. In some embodiments, the cell is isolated from a different individual, modified according to any one of the aforementioned methods, and introduced into a patient. In some embodiments, antibody production in an individual is enhanced by introducing the antibody-producing immune cell modified according to any one of the aforementioned methods to the individual. In some embodiments, the cell is isolated from an individual, modified according to any one of the aforementioned methods, and introduced back into the individual. In some embodiments, the cell is isolated from an individual, modified according to any one of the aforementioned methods, and introduced into a different individual. In some embodiments, the method further comprises the step of contacting the cell with an electric field generated by at least one electrode. Certain aspects of the present invention relate to a system comprising the constriction, cell suspension, and compound for use in any one of the aforementioned methods. In some embodiments, the system further comprises at least one electrode to generate an electric field.

Certain aspects of the present invention provides a method for inducing de novo antibody production in a cell, the method comprising passing a cell suspension through a constriction, wherein said constriction deforms the cell thereby causing a perturbation of the cell such that a compound that initiates antibody productions enters the cell, wherein de novo antibody production in said cell is induced. In some embodiments, the constriction is contained within a microfluidic channel. In some embodiments, the constriction is a pore or contained within a pore. In some embodiments, the pore is contained in a surface. In some embodiments, the surface is a filter. In some embodiments, the surface is a membrane. In some embodiments, the constriction size is a function of the cell diameter. In some embodiments, the constriction size is about 20%, about 30%, about 40%, about 50%, about 60%, about 70%, about 80%, about 90%, or about 99% of the cell diameter.

In some embodiments that can be combined with the previous embodiments, the cell suspension comprises a mixed cell population. In some embodiments, the cell suspension is whole blood. In some embodiments, the cell suspension comprises a purified cell population. In some embodiments, the cell suspension comprises mammalian cells. In some embodiments, the cell suspension comprises monkey, mouse, dog, cat, horse, rat, sheep, goat, or rabbit cells. In some embodiments, the cell suspension comprises human cells. In some embodiments, the cell suspension comprises non-mammalian cells. In some embodiments, the cell suspension comprises bacteria, yeast, chicken, frog, insect, or nematode cells. In some embodiments, the cell suspension comprises peripheral blood mononuclear cells. In some embodiments, the cell is an immune cell, stem cell, bone marrow-derived progenitor cell, erythrocyte precursor, fibroblast, cardiac cell, or cell line cell. In some embodiments, the cell is a B cell, T cell, monocyte, macrophage, neutrophil, eosinophil, dendritic cell, basophil, NK cell, NKT cell, mast cell, or stem cell. In some embodiments, the cell is a B cell or B-cell precursor. In some embodiments, the cell is a B cell precursor, naïve B cell, activated B cell, memory B cell, plasma cell, B-1 cell, marginal-zone B cell, follicular B cell, regulatory B cell, or B cell lymphoma cell. In some embodiments, the cell is a bone-marrow derived B cell precursor.

In some embodiments that can be combined with the previous embodiments, the compound comprises a nucleic acid. In some embodiments, the nucleic acid encodes an immunoglobulin. In some embodiments, the nucleic acid encodes a de novo antibody. In some embodiments, the nucleic acid is integrated into the cell genome. In some embodiments, the nucleic acid is not integrated into the cell genome. In some embodiments, nucleic acid encodes a siRNA, mRNA, miRNA, lncRNA, tRNA, saRNA or shRNA. In some embodiments, the nucleic acid is a plasmid. In some embodiments, the compound comprises a peptide nucleic acid (PNA). In some embodiments, the compound comprises a protein-nucleic acid complex. In some embodiments, the protein-nucleic acid complex comprises a Cas9 protein and a guide RNA. In some embodiments, the protein-nucleic acid complex further comprises donor DNA. In some embodiments, the nucleic acid encodes a Cas9 protein and a guide RNA. In some embodiments, the protein-nucleic acid complex further comprises donor DNA.

In some embodiments that can be combined with the previous embodiments, the compound comprises a protein or polypeptide. In some embodiments, the protein is a TALEN protein, Zinc finger nuclease, mega nuclease, or CRE recombinase. In some embodiments, the protein is a transcription factor. In some embodiments, the protein is a transposase or integrase enzyme.

In some embodiments that can be combined with the previous embodiments, the compound is B-cell activating factor. In some embodiments, the compound is a proliferation inducing ligand. In some embodiments, the compound is an activator of a B cell receptor signaling molecule. In some embodiments, the compound is a cell activation factor. In some embodiments, the compound is a cell differentiation factor. In some embodiments, the compound is a small molecule. In some embodiments, the compound is in a nanoparticle. In some embodiments, the compound is in a liposome. In some embodiments, the compound is in a nucleic acid delivery vehicle. In some embodiments, the compound is in a virus. In some embodiments, the compound is in a viral particle. In some embodiments, the compound is in a vehicle comprising viral capsid. In some embodiments, the compound is in an adeno-associated virus. In some embodiments, the compound is in an adeno-associated virus particle. In some embodiments, the compound is in a vehicle comprising adeno-associated virus capsid.

In some embodiments that can be combined with the previous embodiments, the antibody is a human or humanized antibody. In some embodiments, the antibody is an antigen binding antibody variant. In some embodiments, the antibody class is IgM, IgG, IgA, IgE, or IgD. In some embodiments, the antibody is an antigen binding antibody fragment. In some embodiments, the antibody is a Fab, Fab′, Fab′-SH, Fab₂, F(ab′)₂, Fv, scFv, scFab, or dsFv fragment. In some embodiments, the antibody is a full length antibody. In some embodiments, the antibody is a single-domain antibody. In some embodiments, the antibody is a nanobody, V_{H}H or V_{NAR} antibody fragment. In some embodiments, the antibody is a single-chain antibody. In some embodiments, the antibody is a multi-specific antibody. In some embodiments, the antibody is an antibody fusion protein.

In some embodiments that can be combined with the previous embodiments, said cell suspension is contacted with the compound before, concurrently, or after passing through the constriction. In some embodiments, the channel comprises a constriction length of about 30 μm and a constriction width of about 4 μm. In some embodiments, the channel comprises a constriction length of about 10 μm and a constriction width of about 4 μm. In some embodiments, the pore size is about 0.4 μm, about 4 μm, about 5 μm, about 8 μm, about 10 μm, about 12 μm, or about 14 μm. In some embodiments, the method is performed between about −5° C. and about 45° C.

In some embodiments that can be combined with the previous embodiments, a patient is treated by introducing the cell modified according to any one of the aforementioned methods to the patient. In some embodiments, the cell is isolated from a patient, modified according to any one of the aforementioned methods, and introduced back into the patient. In some embodiments, the cell is isolated from a different individual, modified according to any one of the aforementioned methods, and introduced into a patient. In some embodiments, de novo antibody production in an individual is induced by introducing the cell modified according to any one of the aforementioned methods to the individual. In some embodiments, the cell is isolated from an individual, modified according to any one of the aforementioned methods, and introduced back into the individual. In some embodiments, the cell is isolated from an individual, modified according to any one of the aforementioned methods, and introduced into a different individual. In some embodiments, the method further comprises the step of contacting the cell with an electric field generated by at least one electrode. Certain aspects of the present invention relate to a system comprising the constriction, cell suspension, and compound for use in any one of the aforementioned methods. In some embodiments, the system further comprises at least one electrode to generate an electric field.

### DETAILED DESCRIPTION

The invention provides methods of modulating antibody production by passing a cell suspension through a constriction, enabling delivery of a compound that modulates antibody production to a cell. The invention provides methods for altering endogenous antibody production in an antibody-producing cell, by passing a cell suspension containing the antibody-producing cell through a constriction, wherein the constriction deforms the cell thereby causing a perturbation of the cell such that a compound that alters antibody production enters the antibody-producing cell, wherein endogenous antibody production in the antibody-producing cell is altered. The invention also provides methods for inducing de novo antibody production in a cell by passing a cell suspension through a constriction, wherein the constriction deforms the cell thereby causing a perturbation of the cell such that a compound that initiates antibody productions enters the cell, wherein de novo antibody production in the cell is induced. For example, nucleic acid encoding an antibody may be delivered to a cell capable of producing an antibody.

### I. General Techniques

The techniques and procedures described or referenced herein are generally well understood and commonly employed using conventional methodology by those skilled in the art, such as, for example, the widely utilized methodologies described in Molecular Cloning: A Laboratory Manual (Sambrook et al., 4^{th} ed., Cold Spring Harbor Laboratory Press, Cold Spring Harbor, N.Y., 2012); Current Protocols in Molecular Biology (F. M. Ausubel, et al. eds., 2003); the series Methods in Enzymology (Academic Press, Inc.); PCR 2: A Practical Approach (M. J. MacPherson, B. D. Hames and G. R. Taylor eds., 1995); Antibodies, A Laboratory Manual (Harlow and Lane, eds., 1988); Culture of Animal Cells: A Manual of Basic Technique and Specialized Applications (R. I. Freshney, 6^{th} ed., J. Wiley and Sons, 2010); Oligonucleotide Synthesis (M. J. Gait, ed., 1984); Methods in Molecular Biology, Humana Press; Cell Biology: A Laboratory Notebook (J. E. Cellis, ed., Academic Press, 1998); Introduction to Cell and Tissue Culture (J. P. Mather and P. E. Roberts, Plenum Press, 1998); Cell and Tissue Culture: Laboratory Procedures (A. Doyle, J. B. Griffiths, and D. G. Newell, eds., J. Wiley and Sons, 1993-8); Handbook of Experimental Immunology (D. M. Weir and C. C. Blackwell, eds., 1996); Gene Transfer Vectors for Mammalian Cells (J. M. Miller and M. P. Calos, eds., 1987); PCR: The Polymerase Chain Reaction, (Mullis et al., eds., 1994); Current Protocols in Immunology (J. E. Coligan et al., eds., 1991); Short Protocols in Molecular Biology (Ausubel et al., eds., J. Wiley and Sons, 2002); Immunobiology (C. A. Janeway et al., 2004); Antibodies (P. Finch, 1997); Antibodies: A Practical Approach (D. Catty, ed., IRL Press, 1988-1989); Monoclonal Antibodies: A Practical Approach (P. Shepherd and C. Dean, eds., Oxford University Press, 2000); Using Antibodies: A Laboratory Manual (E. Harlow and D. Lane, Cold Spring Harbor Laboratory Press, 1999); The Antibodies (M. Zanetti and J. D. Capra, eds., Harwood Academic Publishers, 1995); and Cancer: Principles and Practice of Oncology (V. T. DeVita et al., eds., J.B. Lippincott Company, 2011).

### II. Definitions

For purposes of interpreting this specification, the following definitions will apply and whenever appropriate, terms used in the singular will also include the plural and vice versa. In the event that any definition set forth below conflicts with any document incorporated herein by reference, the definition set forth shall control.

As used herein, the singular form “a”, “an”, and “the” includes plural references unless indicated otherwise.

It is understood that aspects and embodiments of the invention described herein include “comprising,” “consisting,” and “consisting essentially of” aspects and embodiments.

The term “about” as used herein refers to the usual error range for the respective value readily known to the skilled person in this technical field. Reference to “about” a value or parameter herein includes (and describes) embodiments that are directed to that value or parameter per se.

The term “antibody” includes monoclonal antibodies (including full length antibodies which have an immunoglobulin Fc region), antibody compositions with polyepitopic specificity, multispecific antibodies (e.g., bispecific antibodies, diabodies, and single-chain molecules), monovalent antibodies, as well as antibody fragments (e.g., Fab, F(ab′)₂, and Fv). The term “immunoglobulin” (Ig) is used interchangeably with “antibody” herein. In some examples, the antibody may be fused to another polypeptide (e.g., a toxic polypeptide, a reporter polypeptide, etc.).

The terms “full-length antibody,” “intact antibody” or “whole antibody” are used interchangeably to refer to an antibody in its substantially intact form, as opposed to an antibody fragment. Specifically, whole antibodies include those with heavy and light chains including an Fc region. The constant domains may be native sequence constant domains (e.g., human native sequence constant domains) or amino acid sequence variants thereof. In some cases, the intact antibody may have one or more effector functions.

The term “antibody fragment” refers to a portion of an intact antibody; for example the antigen binding and/or the variable region of the intact antibody. Examples of antibody fragments include Fab, Fab′, F(ab′)2 and Fv fragments; diabodies; linear antibodies; single-chain antibody molecules and multispecific antibodies formed from antibody fragments.

The term “single-chain antibody” or “single-chain variable fragment”, also abbreviated as “sFv” or “scFv”, refers to antibody fragments that comprise the VH and VL antibody domains connected into a single polypeptide chain. In some embodiments, the sFv polypeptide further comprises a polypeptide linker between the VH and VL domains which enables the sFv to form the desired structure for antigen binding.

The term “single-domain antibody” or “nanobody”, also abbreviated as “sdAb” or “V_{H}H”, refers to an antibody fragment consisting of a single monomeric variable antibody domain.

The term “humanized” refers to forms of non-human (e.g., murine) antibodies that are chimeric antibodies containing minimal sequence derived from non-human immunoglobulin. For example, a humanized antibody may be a human immunoglobulin (recipient antibody) in which residues from a hypervariable region (HVR) of the recipient antibody are replaced by residues from a HVR of a non-human species (donor antibody) such as mouse, rat, rabbit or non-human primate having the desired specificity, affinity, and/or capacity. In some instances, framework (“FR”) residues of the human immunoglobulin are replaced by corresponding non-human residues.

The term “human antibody” refers to an antibody that possesses an amino-acid sequence corresponding to that of an antibody produced by a human.

The term “de novo antibody production” refers to production of an antibody that was not previously produced by a particular cell. In some embodiments, the antibody produced by a cell has a different antigen-binding specificity or a different isotype than the antibody or antibodies previously produced by said cell. In some embodiments, the antibody is produced by a cell that did not previously produce antibodies.

The terms “fusion protein” and “fusion polypeptide” refer to a polypeptide having two portions covalently linked together, where each of the portions is a polypeptide having a different properly. In some embodiments, the fusion protein contains an antibody bound to a heterologous protein. The property may be a biological property, such as activity in vitro or in vivo.

The term “pore” as used herein refers to an opening, including without limitation, a hole, tear, cavity, aperture, break, gap, or perforation within a material. In some examples, (where indicated) the term refers to a pore within a surface of the present disclosure. In other examples, (where indicated) a pore can refer to a pore in a cell membrane.

The term “membrane” as used herein refers to a selective barrier or sheet containing pores. The term includes a pliable sheetlike structure that acts as a boundary or lining. In some examples, the term refers to a surface or filter containing pores. This term is distinct from the term “cell membrane”.

The term “filter” as used herein refers to a porous article that allows selective passage through the pores. In some examples the term refers to a surface or membrane containing pores.

The term “heterogeneous” as used herein refers to something which is mixed or not uniform in structure or composition. In some examples the term refers to pores having varied sizes, shapes or distributions within a given surface.

The term “homogeneous” as used herein refers to something which is consistent or uniform in structure or composition throughout. In some examples the term refers to pores having consistent sizes, shapes, or distribution within a given surface.

The term “heterologous” as used herein refers to a molecule which is derived from a different organism. In some examples the term refers to a nucleic acid or protein which is not normally found or expressed within the given organism.

The term “homologous” as used herein refers to a molecule which is derived from the same organism. In some examples the term refers to a nucleic acid or protein which is normally found or expressed within the given organism.

The term “polynucleotide” or “nucleic acid” as used herein refers to a polymeric form of nucleotides of any length, either ribonucleotides or deoxyribonucleotides. Thus, this term includes, but is not limited to, single-, double- or multi-stranded DNA or RNA, genomic DNA, cDNA, DNA-RNA hybrids, or a polymer comprising purine and pyrimidine bases, or other natural, chemically or biochemically modified, non-natural, or derivatized nucleotide bases. The backbone of the polynucleotide can comprise sugars and phosphate groups (as may typically be found in RNA or DNA), or modified or substituted sugar or phosphate groups. Alternatively, the backbone of the polynucleotide can comprise a polymer of synthetic subunits such as phosphoramidates and thus can be an oligodeoxynucleoside phosphoramidate (P—NH2) or a mixed phosphoramidate-phosphodiester oligomer. In addition, a double-stranded polynucleotide can be obtained from the single stranded polynucleotide product of chemical synthesis either by synthesizing the complementary strand and annealing the strands under appropriate conditions, or by synthesizing the complementary strand de novo using a DNA polymerase with an appropriate primer.

The terms “polypeptide” and “protein” are used interchangeably to refer to a polymer of amino acid residues, and are not limited to a minimum length. Such polymers of amino acid residues may contain natural or non-natural amino acid residues, and include, but are not limited to, peptides, oligopeptides, dimers, trimers, and multimers of amino acid residues. Both full-length proteins and fragments thereof are encompassed by the definition. The terms also include post-expression modifications of the polypeptide, for example, glycosylation, sialylation, acetylation, phosphorylation, and the like. Furthermore, for purposes of the present invention, a “polypeptide” refers to a protein which includes modifications, such as deletions, additions, and substitutions (generally conservative in nature), to the native sequence, as long as the protein maintains the desired activity. These modifications may be deliberate, as through site-directed mutagenesis, or may be accidental, such as through mutations of hosts which produce the proteins or errors due to PCR amplification.

For any of the structural and functional characteristics described herein, methods of determining these characteristics are known in the art.

### III. Endogenous Antibody Production

In certain aspects, the invention provides methods for altering endogenous antibody production in an antibody-producing cell, the methods comprising passing a cell suspension comprising the antibody-producing cell through a constriction, wherein the constriction deforms the cell thereby causing a perturbation of the cell such that a compound that alters antibody production enters the antibody-producing cell, wherein endogenous antibody production in the antibody-producing cell is altered. In some embodiments, the endogenous antibody production is increased or enhanced. In some embodiments, the endogenous antibody production is decreased or downregulated.

In some embodiments, the antibody is a human or humanized antibody. In some embodiments, the antibody is an antigen binding antibody variant. In some embodiments, the antibody class is IgM, IgG, IgA, IgE, or IgD. In some embodiments, the antibody is an antigen binding antibody fragment. In some embodiments, the antibody is a Fab, Fab′, Fab′-SH, Fab₂, F(ab′)₂, Fv, scFv, scFab, or dsFv fragment. In some embodiments, the antibody is a full length antibody. In some embodiments, the antibody is a single-domain antibody. In some embodiments, the antibody is a nanobody, V_{H}H or V_{NAR} antibody fragment. In some embodiments, the antibody is a monovalent antibody. In some embodiments, the antibody is a single-chain antibody. In some embodiments, the antibody is a multi-specific antibody. In some embodiments, the antibody is an antibody fusion protein.

In some embodiments, the endogenous antibody production is altered by at least about 25%, about 50%, about 75%, about 100%, about 150%, about 200%, or more than about 200%. In some embodiments, the endogenous antibody production is increased as compared to the level of antibody produced by the cell prior to passing through the constriction. In some embodiments, the total endogenous antibody production produced by a population of cells is increased. In some embodiments, the endogenous antibody production is decreased as compared to the level of antibody produced by the cell prior to passing through the constriction. In some embodiments, the total endogenous antibody production produced by a population of cells is decreased. In some embodiment, the endogenous antibody production in altered on a per cell basis (i.e. individual cells produce altered antibody levels). In some embodiments, the endogenous antibody production is altered as compared to the cell or cell population's level of antibody production before passing through the device. In some embodiment, enhanced antibody production refers to a more sustained or longer duration of antibody production over time. In some embodiments, the endogenous antibody production is sustained for about 25%, about 50%, about 75%, about 100%, about 150%, about 200%, or more than about 200% longer than antibody production by cells that did not pass through the constriction. In some embodiments, the duration of endogenous antibody production is decreased by about 25%, about 50%, about 75%, or about 100% as compared to antibody production by cells that did not pass through the constriction.

In some embodiments, the endogenous antibody production in culture media after cells are passed through the constriction ranges from about 0 ng/L to about 1 g/L or any concentration or range of concentrations therebetween. In some embodiments, the endogenous antibody production in culture media after cells are passed through the constriction ranges from about 0 ng/L to about 750 mg/L, about 0 ng/L to about 500 mg/L, about 0 ng/L to about 250 mg/L, about 0 ng/L to about 1 mg/L, about 0 ng/L to about 750 μg/L, about 0 ng/L to about 500 μg/L, about 0 ng/L to about 250 μg/L, about 0 ng/L to about 1 μg/L, about 0 ng/L to about 750 ng/L, about 0 ng/L to about 500 ng/L, about 0 ng/L to about 250 ng/L, about 0 ng/L to about 100 ng/L, about 0 ng/L to about 50 ng/L, about 0 ng/L to about 25 ng/L, about Ong/L to about 10 ng/L, or about 0 ng/L to about 5 ng/L. In some embodiments, the endogenous antibody production in culture media after cells are passed through the constriction ranges from about 5 ng/L to about 1 g/L, about 10 ng/L to about 1 g/L, about 25 ng/L to about 1 g/L, about 50 ng/L to about 1 g/L, about 75 ng/L to about 1 g/L, about 100 ng/L to about 1 g/L, about 250 ng/L to about 1 g/L, about 500 ng/L to about 1 g/L, about 750 ng/L to about 1 g/L, about 1 μg/L to about 1 g/L, about 250 μg/L to about 1 g/L, about 500 μg/L to about 1 g/L, about 750 μg/L to about 1 g/L, about 1 mg/L to about 1 g/L, about 250 mg/L to about 1 g/L, about 500 mg/L to about 1 g/L, or about 750 mg/L to about 1 g/L. In some embodiments, the endogenous antibody production after cells are passed through the constriction ranges from about 0 pg/cell/day to about 100 pg/cell/day or any quantity or range of quantities therebetween. In some embodiments, the endogenous antibody after cells are passed through the constriction production ranges from about 0 pg/cell/day to about 75 pg/cell/day, about 0 pg/cell/day to about 50 pg/cell/day, about 0 pg/cell/day to about 25 pg/cell/day, about 0 pg/cell/day to about 10 pg/cell/day, about 0 pg/cell/day to about 5 pg/cell/day, about 0 pg/cell/day to about 2.5 pg/cell/day, about 0 pg/cell/day to about 1 pg/cell/day, about 0 pg/cell/day to about 0.75 pg/cell/day, about 0 pg/cell/day to about 0.5 pg/cell/day, about 0 pg/cell/day to about 0.25 pg/cell/day, about 0 pg/cell/day to about 0.1 pg/cell/day, or about 0 pg/cell/day to about 0.05 pg/cell/day. In some embodiments, the endogenous antibody production after cells are passed through the constriction ranges from about 0.05 pg/cell/day to about 100 pg/cell/day, about 0.1 pg/cell/day to about 100 pg/cell/day, about 0.25 pg/cell/day to about 100 pg/cell/day, about 0.5 pg/cell/day to about 100 pg/cell/day, about 0.75 pg/cell/day to about 100 pg/cell/day, about 1 pg/cell/day to about 100 pg/cell/day, about 2.5 pg/cell/day to about 100 pg/cell/day, about 5 pg/cell/day to about 100 pg/cell/day, about 10 pg/cell/day to about 100 pg/cell/day, about 25 pg/cell/day to about 100 pg/cell/day, about 50 pg/cell/day to about 100 pg/cell/day, or about 75 pg/cell/day to about 100 pg/cell/day.

Endogenous antibody production can be measured by any method known in the art, including without limitation, any direct or competitive binding assay using techniques such as western blots, radioimmunoassays, ELISA (enzyme linked immnosorbent assay), “sandwich” immunoassays, immunoprecipitation assays, fluorescent immunoassays, and protein A immunoassays. In some embodiments, endogenous antibody production can by measured using chromatographic methods such as high performance liquid chromatography (HPLC) or size exclusion chromatography (SEC).

In some embodiments, the invention provides methods for altering endogenous antibody production in an antibody-producing cell wherein the cell is a mammalian cell. In some embodiments, the cell is a monkey, mouse, dog, cat, horse, rat, sheep, goat or rabbit cell. In some embodiments, the cell is a human cell. In some embodiments, the cell suspension comprises non-mammalian cells. In some embodiments, the cell suspension comprises bacteria, yeast, chicken, frog, insect, or nematode cells. In some embodiments, the cell was previously engineered to produce an antibody; for example, a bacterial cell engineered to produce an antibody.

The cell suspension may be a mixed or purified population of cells. In some embodiments, the cell suspension is a mixed cell population, such as whole blood, lymph, and/or peripheral blood mononuclear cells (PBMCs). In some embodiments, the cell suspension is a purified cell population. In some embodiments, the antibody-producing cell is a primary cell or a cell line cell (e.g., an immortalized cell line). In some embodiments, the cell is a hybridoma. In some embodiments, the antibody-producing cell is a blood cell. In some embodiments, the blood cell is an antibody-producing immune cell. In some embodiments, the antibody-producing immune cell is a lymphocyte. In some embodiments, the antibody-producing cell is a B cell or B cell precursor. In some embodiments, the antibody-producing cell is a B cell precursor, naïve B cell, activated B cell, memory B cell, plasma cell, B-1 cell, marginal-zone B cell, follicular B cell, regulatory B cell, or B cell lymphoma cell. In some embodiments, the antibody-producing cell is a bone-marrow derived B cell precursor.

### IV. De Novo Antibody Production

In certain aspects, the invention provides methods for inducing de novo antibody production in a cell, the methods comprising passing a cell suspension through a constriction, wherein the constriction deforms the cell thereby causing a perturbation of the cell such that a compound that initiates antibody productions enters the cell, wherein de novo antibody production in the cell is induced.

In some embodiments, the antibody is a human or humanized antibody. In some embodiments, the antibody is an antigen binding antibody variant. In some embodiments, the antibody class is IgM, IgG, IgA, IgE, or IgD. In some embodiments, the antibody is an antigen binding antibody fragment. In some embodiments, the antibody is a Fab, Fab′, Fab′-SH, Fab₂, F(ab′)₂, Fv, scFv, scFab, or dsFv fragment. In some embodiments, the antibody is a full length antibody. In some embodiments, the antibody is a single-domain antibody. In some embodiments, the antibody is a nanobody, V_{H}H or V_{NAR} antibody fragment. In some embodiments, the antibody is a monovalent antibody. In some embodiments, the antibody is a single-chain antibody. In some embodiments, the antibody is a multi-specific antibody. In some embodiments, the antibody is an antibody fusion protein.

In some embodiments, the de novo antibody production in culture media after cells are passed through the constriction ranges from about 10 ng/L to about 1 g/L or any concentration or range of concentrations therebetween. In some embodiments, the de novo antibody production in culture media after cells are passed through the constriction ranges from about 10 ng/L to about 750 mg/L, about 10 ng/L to about 500 mg/L, about 10 ng/L to about 250 mg/L, about 10 ng/L to about 1 mg/L, about 10 ng/L to about 750 μg/L, about 10 ng/L to about 500 μg/L, about 10 ng/L to about 250 μg/L, about 10 ng/L to about 1 μg/L, about 10 ng/L to about 750 ng/L, about 10 ng/L to about 500 ng/L, about 10 ng/L to about 250 ng/L, about 10 ng/L to about 100 ng/L, about 10 ng/L to about 50 ng/L, or about 10 ng/L to about 25 ng/L. In some embodiments, the de novo antibody production in culture media after cells are passed through the constriction ranges from about 25 ng/L to about 1 g/L, about 50 ng/L to about 1 g/L, about 75 ng/L to about 1 g/L, about 100 ng/L to about 1 g/L, about 250 ng/L to about 1 g/L, about 500 ng/L to about 1 g/L, about 750 ng/L to about 1 g/L, about 1 μg/L to about 1 g/L, about 250 μg/L to about 1 g/L, about 500 μg/L to about 1 g/L, about 750 μg/L to about 1 g/L, about 1 mg/L to about 1 g/L, about 250 mg/L to about 1 g/L, about 500 mg/L to about 1 g/L, or about 750 mg/L to about 1 g/L.

In some embodiments, the de novo antibody production after cells are passed through the constriction ranges from about 0.1 pg/cell/day to about 100 pg/cell/day or any quantity or range of quantities therebetween. In some embodiments, the de novo antibody production after cells are passed through the constriction ranges from about 0.1 pg/cell/day to about 75 pg/cell/day, about 0.1 pg/cell/day to about 50 pg/cell/day, about 0.1 pg/cell/day to about 25 pg/cell/day, about 0.1 pg/cell/day to about 10 pg/cell/day, about 0.1 pg/cell/day to about 5 pg/cell/day, about 0.1 pg/cell/day to about 2.5 pg/cell/day, about 0.1 pg/cell/day to about 1 pg/cell/day, about 0.1 pg/cell/day to about 0.75 pg/cell/day, about 0.1 pg/cell/day to about 0.5 pg/cell/day, or about 0.1 pg/cell/day to about 0.25 pg/cell/day. In some embodiments, the de novo antibody production after cells are passed through the constriction ranges from about 0.25 pg/cell/day to about 100 pg/cell/day, about 0.5 pg/cell/day to about 100 pg/cell/day, about 0.75 pg/cell/day to about 100 pg/cell/day, about 1 pg/cell/day to about 100 pg/cell/day, about 2.5 pg/cell/day to about 100 pg/cell/day, about 5 pg/cell/day to about 100 pg/cell/day, about 10 pg/cell/day to about 100 pg/cell/day, about 25 pg/cell/day to about 100 pg/cell/day, about 50 pg/cell/day to about 100 pg/cell/day, or about 75 pg/cell/day to about 100 pg/cell/day.

De novo antibody production can be measured by any method known in the art, including without limitation, any direct or competitive binding assay using techniques such as western blots, radioimmunoassays, ELISA (enzyme linked immnosorbent assay), “sandwich” immunoassays, immunoprecipitation assays, fluorescent immunoassays, and protein A immunoassays. In some embodiments, de novo antibody production can by measured using chromatographic methods such as high performance liquid chromatography (HPLC) or size exclusion chromatography (SEC).

In some embodiments, the invention provides methods for inducing de novo antibody production in a cell wherein the cell is a mammalian cell. In some embodiments, the cell is a monkey, mouse, dog, cat, horse, rat, sheep, goat or rabbit cell. In some embodiments, the cell is a human cell. In some embodiments, the cell suspension comprises non-mammalian cells. In some embodiments, the cell suspension comprises bacteria, yeast, chicken, frog, insect, or nematode cells.

The cell suspension may be a mixed or purified population of cells. In some embodiments, the cell suspension is a mixed cell population, such as whole blood, lymph, and/or peripheral blood mononuclear cells (PBMCs). In some embodiments, the cell suspension is a purified cell population. In some embodiments, the cell is a primary cell or a cell line cell. In some embodiments, the cell is a blood cell. In some embodiments, the blood cell is an immune cell. In some embodiments, the immune cell is a lymphocyte. In some embodiments, the immune cell is a B cell, T cell, monocyte, macrophage, neutrophil, eosinophil, dendritic cell, basophil, NK cell, NKT cell, mast cell, or neutrophil. In some embodiments, the cell is a B cell or B-cell precursor. In some embodiments, the cell is a B cell precursor, naïve B cell, activated B cell, memory B cell, plasma cell, B-1 cell, marginal-zone B cell, follicular B cell, regulatory B cell, or B cell lymphoma cell. In some embodiments, the cell is a bone-marrow derived B cell precursor.

In some embodiments, the cell is a stem cell. Exemplary stem cells include, without limitation, induced pluripotent stem cells (iPSCs), embryonic stem cells (ESCs), liver stem cells, cardiac stem cells, neural stem cells, and hematopoietic stem cells. In some embodiments, the cell is a bone marrow-derived progenitor cell or an erythrocyte precursor. In some embodiments, the cell is a differentiated cell derived from an iPSC or an ESC. In some embodiments, the cell is a differentiated cell derived from a hematopoietic stem cell (e.g., a B cell differentiated from a hematopoietic stem cell). In some embodiments, the cell is a fibroblast cell, such as a primary fibroblast or newborn human foreskin fibroblast (Nuff cell). In some embodiments, the cell is an immortalized cell line cell, such as a HEK293 cell or a CHO cell. In some embodiments, the cell is a skin cell. In some embodiments, the cell is a cardiac cell. In some embodiments, the cell is a reproductive cell such as an oocyte, ovum, or zygote. In some embodiments, the cell is a neuron. In some embodiments, the cell is a cluster of cells, such as an embryo, given that the cluster of cells is not disrupted when passing through the constriction.

### V. Microfluidic Channels to Provide Cell-Deforming Constrictions

In some embodiments, the invention provides methods for altering antibody production or inducing de novo production of antibodies by passing a cell suspension through a constriction, wherein the constriction deforms the cell thereby causing a perturbation of the cell such that a compound that modulates antibody production enters the cell, wherein the constriction is contained within a microfluidic channel. In some embodiments, the endogenous antibody production is enhanced. In some embodiments, the endogenous antibody production is decreased. In some embodiments, multiple constrictions can be placed in parallel and/or in series within the microfluidic channel. Exemplary microfluidic channels containing cell-deforming constrictions for use in the methods disclosed herein are described in WO2013059343.

In some embodiments, the microfluidic channel includes a lumen and is configured such that a cell suspended in a buffer can pass through, wherein the microfluidic channel includes a constriction. The microfluidic channel can be made of any one of a number of materials, including silicon, metal (e.g., stainless steel), plastic (e.g., polystyrene), ceramics, glass, crystalline substrates, amorphous substrates, or polymers (e.g., Poly-methyl methacrylate (PMMA), PDMS, Cyclic Olefin Copolymer (COC), etc.). Fabrication of the microfluidic channel can be performed by any method known in the art, including dry etching, wet etching, photolithography, injection molding, laser ablation, or SU-8 masks.

In some embodiments, the constriction within the microfluidic channel includes an entrance portion, a centerpoint, and an exit portion. In some embodiments, the length, depth, and width of the constriction within the microfluidic channel can vary. In some embodiments, the diameter of the constriction within the microfluidic channel is a function of the diameter of the cell or cluster of cells. In some embodiments, the diameter of the constriction within the microfluidic channel is about 20% to about 99% of the diameter of the cell. In some embodiments, the constriction size is about 20%, about 30%, about 40%, about 50%, about 60%, about 70%, about 80%, about 90%, or about 99% of the cell diameter. In some embodiments, the channel comprises a constriction length of about 30 um and a constriction width of about 4 um. In some embodiments, the channel comprises a constriction length of about 10 um and a constriction width of about 4 um. The cross-section of the channel, the entrance portion, the centerpoint, and the exit portion can also vary. For example, the cross-sections can be circular, elliptical, an elongated slit, square, hexagonal, or triangular in shape. The entrance portion defines a constriction angle, wherein the constriction angle is optimized to reduce clogging of the channel and optimized for enhanced delivery of a compound into the cell. The angle of the exit portion can vary as well. For example, the angle of the exit portion is configured to reduce the likelihood of turbulence that can result in non-laminar flow. In some embodiments, the walls of the entrance portion and/or the exit portion are linear. In other embodiments, the walls of the entrance portion and/or the exit portion are curved.

### VI. Surface Having Pores to Provide Cell Deforming Constrictions

In some embodiments, the invention provides methods for altering antibody production or inducing de novo production of antibodies by passing a cell suspension through a constriction, wherein the constriction deforms the cell thereby causing a perturbation of the cell such that a compound that modulates antibody production enters the cell, wherein the constriction is a pore or contained within a pore. In some embodiments, the pore is contained in a surface. Exemplary surfaces having pores for use in the methods disclosed herein are described in U.S. Provisional Application 62/214,820, filed Sep. 4, 2015.

The surfaces as disclosed herein can be made of any one of a number of materials and take any one of a number of forms. In some embodiments, the surface is a filter. In some embodiments, the surface is a membrane. In some embodiments, the filter is a tangential flow filter. In some embodiments, the surface is a sponge or sponge-like matrix. In some embodiments, the surface is a matrix.

In some embodiments the surface is a tortuous path surface. In some embodiments, the tortuous path surface comprises cellulose acetate. In some embodiments, the surface comprises a material selected from, without limitation, synthetic or natural polymers, polycarbonate, silicon, glass, metal, alloy, cellulose nitrate, silver, cellulose acetate, nylon, polyester, polyethersulfone, Polyacrylonitrile (PAN), polypropylene, PVDF, polytetrafluorethylene, mixed cellulose ester, porcelain, and ceramic.

The surface disclosed herein can have any shape known in the art; e.g. a 3-dimensional shape. The 2-dimensional shape of the surface can be, without limitation, circular, elliptical, round, square, star-shaped, triangular, polygonal, pentagonal, hexagonal, heptagonal, or octagonal. In some embodiments, the surface is round in shape. In some embodiments, the surface 3-dimensional shape is cylindrical, conical, or cuboidal.

The surface can have various cross-sectional widths and thicknesses. In some embodiments, the surface cross-sectional width is between about 1 mm and about 1 m or any cross-sectional width or range of cross-sectional widths therebetween. In some embodiments, the surface has a defined thickness. In some embodiments, the surface thickness is uniform. In some embodiments, the surface thickness is variable. For example, in some embodiments, portions of the surface are thicker or thinner than other portions of the surface. In some embodiments, the surface thickness varies by about 1% to about 90% or any percentage or range of percentages therebetween. In some embodiments, the surface is between about 0.01 μm to about 5 mm thick or any thickness or range of thicknesses therebetween.

In some embodiments, the constriction is a pore or contained within a pore. The cross-sectional width of the pores is related to the type of cell to be treated. In some embodiments, the pore size is a function of the diameter of the cell of cluster of cells to be treated. In some embodiments, the pore size is such that a cell is perturbed upon passing through the pore. In some embodiments, the pore size is less than the diameter of the cell. In some embodiments, the pore size is about 20% to about 99% of the diameter of the cell. In some embodiments, the pore size is about 20%, about 30%, about 40%, about 50%, about 60%, about 70%, about 80%, about 90%, or about 99% of the cell diameter. Optimal pore size can vary based upon the application and/or cell type. In some embodiments, the pore size is about 0.4 μm, about 4 μm, about 5 μm, about 8 μm, about 10 μm, about 12 μm, or about 14 μm.

The entrances and exits of the pore passage may have a variety of angles. The pore angle can be selected to minimize clogging of the pore while cells are passing through. In some embodiments, the flow rate through the surface is between about 0.001 mL/cm²/sec to about 100 L/cm²/sec or any rate or range of rates therebetween. For example, the angle of the entrance or exit portion can be between about 0 and about 90 degrees. In some embodiments, the pores have identical entrance and exit angles. In some embodiments, the pores have different entrance and exit angles. In some embodiments, the pore edge is smooth, e.g. rounded or curved. A smooth pore edge has a continuous, flat, and even surface without bumps, ridges, or uneven parts. In some embodiments, the pore edge is sharp. A sharp pore edge has a thin edge that is pointed or at an acute angle. In some embodiments, the pore passage is straight. A straight pore passage does not contain curves, bends, angles, or other irregularities. In some embodiments, the pore passage is curved. A curved pore passage is bent or deviates from a straight line. In some embodiments, the pore passage has multiple curves, e.g. about 2, 3, 4, 5, 6, 7, 8, 9, 10 or more curves.

The pores can have any shape known in the art, including a 2-dimensional or 3-dimensional shape. The pore shape (e.g., the cross-sectional shape) can be, without limitation, circular, elliptical, round, square, star-shaped, triangular, polygonal, pentagonal, hexagonal, heptagonal, and octagonal. In some embodiments, the cross-section of the pore is round in shape. In some embodiments, the 3-dimensional shape of the pore is cylindrical or conical. In some embodiments, the pore has a fluted entrance and exit shape. In some embodiments, the pore shape is homogenous (i.e. consistent or regular) among pores within a given surface. In some embodiments, the pore shape is heterogeneous (i.e. mixed or varied) among pores within a given surface.

The surfaces described herein can have a range of total pore numbers. In some embodiments, the pores encompass about 10% to about 80% of the total surface area. In some embodiments, the surface contains about 1.0×10⁵ to about 1.0×10³⁰ total pores or any number or range of numbers therebetween. In some embodiments, the surface comprises between about 10 and about 1.0×10¹⁵ pores per mm² surface area.

The pores can be distributed in numerous ways within a given surface. In some embodiments, the pores are distributed in parallel within a given surface. In one such example, the pores are distributed side-by-side in the same direction and are the same distance apart within a given surface. In some embodiments, the pore distribution is ordered or homogeneous. In one such example, the pores are distributed in a regular, systematic pattern or are the same distance apart within a given surface. In some embodiments, the pore distribution is random or heterogeneous. In one such example, the pores are distributed in an irregular, disordered pattern or are different distances apart within a given surface. In some embodiments, multiple surfaces are distributed in series. The multiple surfaces can be homogeneous or heterogeneous in surface size, shape, and/or roughness. The multiple surfaces can further contain pores with homogeneous or heterogeneous pore size, shape, and/or number, thereby enabling the simultaneous delivery of a range of compounds into different cell types.

In some embodiments, an individual pore has a uniform width dimension (i.e. constant width along the length of the pore passage). In some embodiments, an individual pore has a variable width (i.e. increasing or decreasing width along the length of the pore passage). In some embodiments, pores within a given surface have the same individual pore depths. In some embodiments, pores within a given surface have different individual pore depths. In some embodiments, the pores are immediately adjacent to each other. In some embodiments, the pores are separated from each other by a distance. In some embodiments, the pores are separated from each other by a distance of about 0.001 μm to about 30 mm or any distance or range of distances therebetween.

In some embodiments, the surface is coated with a material. The material can be selected from any material known in the art, including, without limitation, Teflon, an adhesive coating, surfactants, proteins, adhesion molecules, antibodies, anticoagulants, factors that modulate cellular function, nucleic acids, lipids, carbohydrates, or transmembrane proteins. In some embodiments, the surface is coated with polyvinylpyrrolidone. In some embodiments, the material is covalently attached to the surface. In some embodiments, the material is non-covalently attached to the surface. In some embodiments, the surface molecules are released at the cells pass through the pores.

In some embodiments, the surface has modified chemical properties. In some embodiments, the surface is hydrophilic. In some embodiments, the surface is hydrophobic. In some embodiments, the surface is charged. In some embodiments, the surface is positively and/or negatively charged. In some embodiments, the surface can be positively charged in some regions and negatively charged in other regions. In some embodiments, the surface has an overall positive or overall negative charge. In some embodiments, the surface can be any one of smooth, electropolished, rough, or plasma treated. In some embodiments, the surface comprises a zwitterion or dipolar compound. In some embodiments, the surface is plasma treated.

In some embodiments, the surface is contained within a larger module. In some embodiments, the surface is contained within a syringe, such as a plastic or glass syringe. In some embodiments, the surface is contained within a plastic filter holder. In some embodiments, the surface is contained within a pipette tip.

### VII. Cell Perturbations

In some embodiments, the invention provides methods for altering antibody production or inducing de novo production of antibodies by passing a cell suspension through a constriction, wherein the constriction deforms the cell thereby causing a perturbation of the cell such that a compound that modulates antibody productions enters the cell, wherein the perturbation in the cell is a breach in the cell that allows material from outside the cell to move into the cell (e.g., a hole, tear, cavity, aperture, pore, break, gap, perforation). In some embodiments, the endogenous antibody production is enhanced. In some embodiments, the endogenous antibody production is decreased. The deformation can be caused by, for example, pressure induced by mechanical strain and/or shear forces. In some embodiments, the perturbation is a perturbation within the cell membrane. In some embodiments, the perturbation is transient. In some embodiments, the cell perturbation lasts from about 1.0×10⁻⁹ seconds to about 2 hours, or any time or range of times therebetween. In some embodiments, the cell perturbation lasts for about 1.0×10⁻⁹ second to about 1 second, about 1 second to about 1 minute, or about 1 minute to about 1 hour. In some embodiments, the cell perturbation lasts for between any one of about 1.0×10⁻⁹ to about 1.0×10⁻¹, about 1.0×10⁻⁹ to about 1.0×10⁻², about 1.0×10⁻⁹ to about 1.0×10⁻³, about 1.0×10⁻⁹ to about 1.0×10⁻⁴, about 1.0×10⁻⁹ to about 1.0×10⁻⁵, about 1.0×10⁻⁹ to about 1.0×10⁻⁶, about 1.0×10⁻⁹ to about 1.0×10⁻⁷, or about 1.0×10⁻⁹ to about 1.0×10⁻⁸ seconds. In some embodiment, the cell perturbation lasts for any one of about 1.0×10⁻⁸ to about 1.0×10⁻¹, about 1.0×10⁻⁷ to about 1.0×10⁻¹, about 1.0×10⁻⁶ to about 1.0×10⁻¹, about 1.0×10⁻⁵ to about 1.0×10⁻¹, about 1.0×10⁻⁴ to about 1.0×10⁻¹, about 1.0×10⁻³ to about 1.0×10⁻¹, or about 1.0×10⁻² to about 1.0×10⁻¹ seconds. The cell perturbations (e.g., pores or holes) created by the methods described herein are not formed as a result of assembly of protein subunits to form a multimeric pore structure such as that created by complement or bacterial hemolysins.

As the cell passes through the constriction, the constriction temporarily imparts injury to the cell membrane that causes passive diffusion of material through the perturbation. In some embodiments, the cell is only deformed for a brief period of time, on the order of 100 μs to minimize the chance of activating apoptotic pathways through cell signaling mechanisms, although other durations are possible (e.g., ranging from nanoseconds to hours). In some embodiments, the cell is deformed for about 1.0×10⁻⁹ seconds to about 2 hours, or any time or range of times therebetween. In some embodiments, the cell is deformed for about 1.0×10⁻⁹ second to about 1 second, about 1 second to about 1 minute, or about 1 minute to about 1 hour. In some embodiments, the cell is deformed for between any one of about 1.0×10⁻⁹ to about 1.0×10⁻¹, about 1.0×10⁻⁹ to about 1.0×10⁻², about 1.0×10⁻⁹ to about 1.0×10⁻³, about 1.0×10⁻⁹ to about 1.0×10⁻⁴, about 1.0×10⁻⁹ to about 1.0×10⁻⁵, about 1.0×10⁻⁹ to about 1.0×10⁻⁶, about 1.0×10⁻⁹ to about 1.0×10⁻⁷, or about 1.0×10⁻⁹ to about 1.0×10⁻⁸ seconds. In some embodiment, the cell is deformed for any one of about 1.0×10⁻⁸ to about 1.0×10⁻¹, about 1.0×10⁻⁷ to about 1.0×10⁻¹, about 1.0×10⁻⁶ to about 1.0×10⁻¹, about 1.0×10⁻⁵ to about 1.0×10⁻¹, about 1.0×10⁻⁴ to about 1.0×10⁻¹, about 1.0×10⁻³ to about 1.0×10⁻¹, or about 1.0×10⁻² to about 1.0×10⁻¹ seconds. In some embodiments, deforming the cell includes deforming the cell for a time ranging from, without limitation, about 1 μs to at least about 750 μs, e.g., at least about 10 μs, 50 μs, 100 μs, 500 μs, or 750 μs.

In some embodiments, the passage of the compound into the cell occurs simultaneously with the cell passing through the constriction and/or the perturbation of the cell. In some embodiments, passage of the compound into the cell occurs after the cell passes through the constriction. In some embodiments, passage of the compound into the cell occurs on the order of minutes after the cell passes through the constriction. In some embodiments, the passage of the compound into the cell occurs from about 1.0×10⁻² seconds to at least about 30 minutes after the cell passes through the constriction. For example, the passage of the compound into the cell occurs from about 1.0×10⁻² seconds to about 1 second, about 1 second to about 1 minute, or about 1 minute to about 30 minutes after the cell passes through the constriction. In some embodiments, the passage of the compound into the cell occurs about 1.0×10⁻² seconds to about 10 minutes, about 1.0×10⁻² seconds to about 5 minutes, about 1.0×10⁻² seconds to about 1 minute, about 1.0×10⁻² seconds to about 50 seconds, about 1.0×10⁻² seconds to about 10 seconds, about 1.0×10⁻² seconds to about 1 second, or about 1.0×10⁻² seconds to about 0.1 second after the cell passes through the constriction. In some embodiments, the passage of the compound into the cell occurs about 1.0×10⁻¹ seconds to about 10 minutes, about 1 second to about 10 minutes, about 10 seconds to about 10 minute, about 50 seconds to about 10 minutes, about 1 minute to about 10 minutes, or about 5 minutes to about 10 minutes after the cell passes through the constriction. In some embodiments, a perturbation in the cell after it passes through the constriction is corrected within the order of about five minutes after the cell passes through the constriction.

In some embodiments, the cell viability after passing through a constriction is about 5% to about 100%. In some embodiments, the cell viability after passing through the constriction is at least about 5%, 10%, 20%, 30%, 40%, 50%, 60%, 70%, 75%, 80%, 85%, 90%, 95%, or 99%. In some embodiments, the cell viability is measured from about 1.0×10⁻² seconds to at least about 10 days after the cell passes through the constriction. For example, the cell viability is measured from about 1.0×10⁻² seconds to about 1 second, about 1 second to about 1 minute, about 1 minute to about 30 minutes, or about 30 minutes to about 2 hours after the cell passes through the constriction. In some embodiments, the cell viability is measured about 1.0×10⁻² seconds to about 2 hours, about 1.0×10⁻² seconds to about 1 hour, about 1.0×10⁻² seconds to about 30 minutes, about 1.0×10⁻² seconds to about 1 minute, about 1.0×10⁻² seconds to about 30 seconds, about 1.0×10⁻² seconds to about 1 second, or about 1.0×10⁻² seconds to about 0.1 second after the cell passes through the constriction. In some embodiments, the cell viability is measured about 1.5 hours to about 2 hours, about 1 hour to about 2 hours, about 30 minutes to about 2 hours, about 15 minutes to about 2 hours, about 1 minute to about 2 hours, about 30 seconds to about 2 hours, or about 1 second to about 2 hours after the cell passes through the constriction. In some embodiments, the cell viability is measured about 2 hours to about 5 hours, about 5 hours to about 12 hours, about 12 hours to about 24 hours, or about 24 hours to about 10 days after the cell passes through the constriction.

### VIII. Delivery Parameters

A number of parameters may influence the delivery of a compound to a cell for altering antibody production or inducing de novo production of antibodies by the methods described herein. In some embodiments, the endogenous antibody production is enhanced. In some embodiments, the endogenous antibody production is decreased. In some embodiments, the cell suspension is contacted with the compound before, concurrently, or after passing through the constriction. The cell may pass through the constriction suspended in a solution that includes the compound to deliver, although the compound can be added to the cell suspension after the cells pass through the constriction. In some embodiments, the compound to be delivered is coated on the constriction.

Examples of parameters that may influence the delivery of the compound into the cell include, but are not limited to, the dimensions of the constriction, the entrance angle of the constriction, the surface properties of the constrictions (e.g., roughness, chemical modification, hydrophilic, hydrophobic, etc.), the operating flow speeds (e.g., cell transit time through the constriction), the cell concentration, the concentration of the compound in the cell suspension, and the amount of time that the cell recovers or incubates after passing through the constrictions can affect the passage of the delivered compound into the cell. Additional parameters influencing the delivery of the compound into the cell can include the velocity of the cell in the constriction, the shear rate in the constriction, the viscosity of the cell suspension, the velocity component that is perpendicular to flow velocity, and time in the constriction. Such parameters can be designed to control delivery of the compound. In some embodiments, the cell concentration ranges from about 10 to at least about 10¹² cells/ml or any concentration or range of concentrations therebetween. In some embodiments, delivery compound concentrations can range from about 10 ng/ml to about 1 g/mL or any concentration or range of concentrations therebetween. In some embodiments, delivery compound concentrations can range from about 1 pM to at least about 2M or any concentration or range of concentrations therebetween.

The temperature used in the methods of the present disclosure can be adjusted to affect compound delivery and cell viability. In some embodiments, the method is performed between about −5° C. and about 45° C. For example, the methods can be carried out at room temperature (e.g., about 20° C.), physiological temperature (e.g., about 37° C.), higher than physiological temperature (e.g., greater than about 37° C. to 45° C. or more), or reduced temperature (e.g., about −5° C. to about 4° C.), or temperatures between these exemplary temperatures.

Various methods can be utilized to drive the cells through the constrictions. For example, pressure can be applied by a pump on the entrance side (e.g., gas cylinder, or compressor), a vacuum can be applied by a vacuum pump on the exit side, capillary action can be applied through a tube, and/or the system can be gravity fed. Displacement based flow systems can also be used (e.g., syringe pump, peristaltic pump, manual syringe or pipette, pistons, etc.). In some embodiments, the cells are passed through the constrictions by positive pressure or negative pressure. In some embodiments, the cells are passed through the constrictions by constant pressure or variable pressure. In some embodiments, pressure is applied using a syringe. In some embodiments, pressure is applied using a pump. In some embodiments, the pump is a peristaltic pump or a diaphragm pump. In some embodiments, pressure is applied using a vacuum. In some embodiments, the cells are passed through the constrictions by g-force. In some embodiments, the cells are passed through the constrictions by capillary pressure.

In some embodiments, fluid flow directs the cells through the constrictions. In some embodiments, the fluid flow is turbulent flow prior to the cells passing through the constriction. Turbulent flow is a fluid flow in which the velocity at a given point varies erratically in magnitude and direction. In some embodiments, the fluid flow through the constriction is laminar flow. Laminar flow involves uninterrupted flow in a fluid near a solid boundary in which the direction of flow at every point remains constant. In some embodiments, the fluid flow is turbulent flow after the cells pass through the constriction. The velocity at which the cells pass through the constrictions can be varied. In some embodiments, the cells pass through the constrictions at a uniform cell speed. In some embodiments, the cells pass through the constrictions at a fluctuating cell speed.

In other embodiments, a combination treatment is used to alter antibody production or induce de novo antibody production, e.g., the methods described herein followed by exposure to an electric field downstream of the constriction. In some embodiments, the endogenous antibody production is enhanced. In some embodiments, the endogenous antibody production is decreased. In some embodiments, the cell is passed through an electric field generated by at least one electrode after passing through the constriction. In some embodiments, the electric field assists in delivery of compounds that enhance antibody production or induce de novo antibody production to a second location inside the cell such as the cell nucleus. For example, the combination of a cell-deforming constriction and an electric field delivers a plasmid encoding an antibody into the cell (e.g., the cell nucleus), resulting in the de novo production of antibody. In some embodiments, one or more electrodes are in proximity to the cell-deforming constriction to generate an electric field. In some embodiments, the electric field is between about 0.1 kV/m to about 100 MV/m, or any number or range of numbers therebetween. In some embodiments, an integrated circuit is used to provide an electrical signal to drive the electrodes. In some embodiments, the cells are exposed to the electric field for a pulse width of between about 1 ns to about 1 s and a period of between about 100 ns to about 10 s or any time or range of times therebetween.

### IX. Cell Suspensions for the Production of Antibodies

The composition of the cell suspension (e.g., osmolarity, salt concentration, serum content, cell concentration, pH, etc.) can impact delivery of the compound for altering antibody production or inducing de novo antibody production. In some embodiments, the endogenous antibody production is enhanced. In some embodiments, the endogenous antibody production is decreased. In some embodiments, the suspension comprises whole blood. Alternatively, the cell suspension is a mixture of cells in a physiological saline solution or physiological medium other than blood. In some embodiments, the cell suspension comprises an aqueous solution. In some embodiments, the aqueous solution comprises cell culture medium, PBS, salts, sugars, growth factors, animal derived products, bulking materials, surfactants, lubricants, vitamins, amino acids, proteins, and/or an agent that impacts actin polymerization. In some embodiments, the cell culture medium is DMEM, Opti-MEM™, IMDM, or RPMI. Additionally, solution buffer can include one or more lubricants (pluronics or other surfactants) that can be designed, for example, to reduce or eliminate clogging of the surface and improve cell viability. Exemplary surfactants include, without limitation, poloxamer, polysorbates, sugars or sugar alcohols such as mannitol, sorbitol, animal derived serum, and albumin protein.

In some configurations with certain types of cells, the cells can be incubated in one or more solutions that aid in the delivery of the compound to the interior of the cell. In some embodiments, the aqueous solution comprises an agent that impacts actin polymerization. In some embodiments, the agent that impacts actin polymerization is Latrunculin A, Cytochalasin, and/or Colchicine. For example, the cells can be incubated in a depolymerization solution such as Lantrunculin A (0.1 μg/ml) for 1 hour prior to delivery to depolymerize the actin cytoskeleton. As an additional example, the cells can be incubated in 10 μM Colchicine (Sigma) for 2 hours prior to delivery to depolymerize the microtubule network.

In some embodiments, the cell population is an enriched prior to use in the disclosed methods. For example, cells are obtained from a bodily fluid, e.g., peripheral blood, and optionally enriched or purified to concentrate B cells. Cells may be enriched may any methods known in the art, including without limitation, magnetic cell separation, fluorescent activated cell sorting (FACS), or density gradient centrifugation.

The viscosity of the cell suspension can also impact the methods disclosed herein. In some embodiments, the viscosity of the cell suspension ranges from about 8.9×10⁻⁴ Pa·s to about 4.0×10⁻³ Pa·s or any value or range of values therebetween. In some embodiments, the viscosity ranges between any one of about 8.9×10 Pa·s to about 4.0×10⁻³ Pa·s, about 8.9×10⁻⁴ Pa·s to about 3.0×10⁻³ Pa·s, about 8.9×10⁻⁴ Pa·s to about 2.0×10⁻³ Pa·s, or about 8.9×10⁻³ Pa·s to about 1.0×10⁻³ Pa·s. In some embodiments, the viscosity ranges between any one of about 0.89 cP to about 4.0 cP, about 0.89 cP to about 3.0 cP, about 0.89 cP to about 2.0 cP, or about 0.89 cP to about 1.0 cP. In some embodiments, a shear thinning effect is observed, in which the viscosity of the cell suspension decreases under conditions of shear strain. Viscosity can be measured by any method known in the art, including without limitation, viscometers, such as a glass capillary viscometer, or rheometers. A viscometer measures viscosity under one flow condition, while a rheometer is used to measure viscosities which vary with flow conditions. In some embodiments, the viscosity is measured for a shear thinning solution such as blood. In some embodiments, the viscosity is measured between about −5° C. and about 45° C. For example, the viscosity is measured at room temperature (e.g., about 20° C.), physiological temperature (e.g., about 37° C.), higher than physiological temperature (e.g., greater than about 37° C. to 45° C. or more), reduced temperature (e.g., about −5° C. to about 4° C.), or temperatures between these exemplary temperatures.

### X. Compounds to Alter or Induce Antibody Production

In some embodiments the invention provides compounds to alter endogenous antibody production in an antibody-producing cell or induce de novo production of antibodies in a cell, wherein the compound is delivered to the cell by any of the methods described herein. In some embodiments, the endogenous antibody production is enhanced. In some embodiments, the endogenous antibody production is decreased. In some embodiments, the compound is a single compound. In some embodiments, the compound is a mixture of compounds. In some embodiments, the compound comprises a nucleic acid. In some embodiments, the compound is a nucleic acid. Exemplary nucleic acids include, without limitation, recombinant nucleic acids, DNA, recombinant DNA, cDNA, genomic DNA, RNA, siRNA, mRNA, saRNA, miRNA, lncRNA, tRNA, and shRNA. In some embodiments, the nucleic acid is homologous to a nucleic acid in the cell. In some embodiments, the nucleic acid is heterologous to a nucleic acid in the cell. In some embodiments, the nucleic acid is a plasmid.

In some embodiments, the nucleic acid encodes an antibody for de novo antibody production. In some embodiments, the nucleic acid encodes a human or humanized antibody. In some embodiments, the nucleic acid encodes an antigen binding antibody variant. In some embodiments, the nucleic acid encodes an IgM, IgG, IgA, IgE, or IgD antibody. In some embodiments, the nucleic acid encodes an antigen binding antibody fragment. In some embodiments, the nucleic acid encodes a Fab, Fab′, Fab′-SH, Fab₂, F(ab′)₂, Fv, scFv, scFab, or dsFv antibody fragment. In some embodiments, the nucleic acid encodes a full length antibody, single-domain antibody, monovalent antibody, single-chain antibody, multi-specific antibody, or antibody fusion protein. In some embodiments, the nucleic acid encodes a nanobody, V_{H}H or V_{NAR} antibody fragment.

In some embodiments, the compound comprises a protein-nucleic acid complex. In some embodiments, the compound is a protein-nucleic acid complex. In some embodiments, protein-nucleic acid complexes, such as clustered regularly interspaced short palindromic repeats (CRISPR)-Cas9, are used in genome editing applications. These complexes contain sequence-specific DNA-binding domains in combination with nonspecific DNA cleavage nucleases. These complexes enable targeted genome editing, including adding, disrupting, or changing the sequence of a specific gene. In some embodiments, a disabled CRISPR is used to block or induce transcription of a target gene. In some embodiments, the protein-nucleic acid complex contains a Cas9 protein and a guide RNA. In some embodiments, the protein-nucleic acid complex further comprises donor DNA for homologous recombination. In some embodiments, the compound includes a nucleic acid encoding for a Cas9 protein and a guide RNA. In some embodiments, the Cas9 protein and guide RNA are on the same plasmid construct. In some embodiments, the Cas9 protein and guide RNA are on different plasmid constructs. In some embodiments, the compound further includes nucleic acid encoding for a donor DNA for homologous recombination. In some embodiments, enzymes such as transposase or integrase are delivered to mediate nucleic acid integration. In some embodiments, delivery of gene editing components by the methods disclosed herein can be used to alter expression of antibodies involved in mediating diseases. For example, delivery of CRISPR compounds can be used to inhibit expression of auto-reactive antibodies that mediate autoimmune diseases.

In some embodiments, the compound comprises a protein or polypeptide. In some embodiments, the compound is a protein or polypeptide. In some embodiments, the protein is a gene-editing protein or nuclease such as a zinc-finger nuclease (ZFN), transcription activator-like effector nuclease (TALEN), mega nuclease, or CRE recombinase. In some embodiments, the protein is a transcription factor. Exemplary transcription factors include, without limitation, BLIMP1, XBP1, IRF4, and BCL-6.

In some embodiments, the protein is an anti-apoptotic protein. Exemplary anti-apoptotic proteins include, without limitation, Bcl-2 family proteins. The anti-apoptotic Bcl-2 proteins include Bcl-2 itself, Bcl-XL, Bcl-w, MC1-1, A1 and Diva. In some embodiments, the protein is a B-cell activating factor. Exemplary B-cell activating and proliferation inducing factors include, without limitation, a proliferation-inducing ligand (APRIL) and B cell activating factor (BAFF). B cell activating factor (BAFF), also known as BLyS, TALL-1, THANK, zTNF4 or TNFSF-13B, is a member of the TNF family and initiates downstream signaling pathways and regulates of B cell survival, maturation and differentiation by binding to its homologous receptors. BAFF and three additional ligands (APRIL, EDA and TWEAK) have similar function and structure characteristics. BAFF and APRIL are potent stimulators of B-cell maturation, proliferation and survival. The three receptors of BAFF, including BCMA (B cell maturation antigen), TACI (transmembrane activator and CAML interactor), and BAFF-R (BAFF receptor, Br3), are all transmembrane proteins. BAFF and APRIL are capable of binding to TACI and BCMA with high affinity, and BAFF can also bind to BAFF-R. Besides the function of promoting B cell survival, BAFF also plays a role in the regulation of germinal centers, isotype switching, and T cell activation.

In some embodiments, the compound is an activator of a B cell receptor signaling molecule. Exemplary B cell receptor signaling molecules include, without limitation, kinases such as Src, Syk, Btk, Erk, Akt, and JNK, adaptor proteins such as CD19 and BLNK, enzymes such as PLC and PIK3, GTPases, and nuclear factors such as NFkB and Ap-1. In some embodiments, the compound is ATP. In some embodiments, the compound is a cell activation factor. In some embodiments, the compound is a nucleic acid encoding for a cell activation factor. Exemplary cell activation factors, include, without limitation, TLR agonists such as CPG and LPS, CD40, CD21, CD19, and CD81. For example, CD19 is involved in B-cell receptor signaling and lowers the threshold for antigen receptor stimulation of B cells. In some embodiments, the compound is a factor which recruits CD4 T cell help, including without limitation chemokines, cytokines, or adhesion molecules.

In some embodiments, the compound is a factor that alters a cellular differentiation state. In some embodiments the factor that alters a cellular differentiation state is a cell differentiation factor. Exemplary cell differentiation factors include, without limitation, CXCL12, FLT3L, IL-7, SCF, RANKL, and neuroleukin. B cell development and differentiation are tightly regulated by lineage specific growth factors and cell adhesion molecules. Interleukin 7 (IL-7), secreted by stromal cells, is an important growth factor for early B cell development and can stimulate pro and pre B cell proliferation. IL-7 dependent pro-B cell proliferation is potentiated by two stromal growth factors, insulin like growth factor-1 (IGF-1) and stem cell factor (SCF). The stromal cell-derived factor 1 or pre-B cell growth-stimulating factor (SDF-1/PBSF), produced by bone marrow stromal cells, induces proliferation of pro and pre-B cells. IL-3 stimulates pre-B cell proliferation through the interaction with IL-3 receptor on B cells, and together with IL-6, IL-3 can stimulate multipotential stem cells and B cell progenitors. Neuroleukin, a glucose-6-phosphate isomer homolog, also has the ability to stimulate B cell development. In some embodiments, the factor that alters a cellular differentiation state is a reprogramming factor. In some embodiments, delivery of a reprogramming factor results in conversion of an antibody-producing cell to a less differentiated state (e.g., conversion of a plasma cell to a memory B cell). In some embodiments, delivery of a reprogramming factor results in antibody class switching. For example, delivery of a reprogramming factor results in class switching from an IgG antibody to an IgA antibody. Delivery of a reprogramming factor to a cell by the methods disclosed herein can result in altered antibody specificity, functional activity, or secretion.

In some embodiments, the compound comprises a small molecule. In some embodiments, the compound is a small molecule. Exemplary small molecules include, without limitation, pharmaceutical agents, metabolities, or radionucleotides. In some embodiments, the pharmaceutical agent is a therapeutic drug.

In some embodiments, the compound is in a nanoparticle. Examples of nanoparticles include gold nanoparticles, quantum dots, carbon nanotubes, nanoshells, dendrimers, and liposomes. In some embodiments, the nanoshells comprise natural or synthetic polymers. In some embodiments, the compound is in a liposome. In some embodiments, the nanoparticle contains a therapeutic molecule. In some embodiments, the nanoparticle contains a nucleic acid, such as mRNA.

In some embodiments, the compound to deliver is purified. In some embodiments, the compound is at least about 60% by weight (dry weight) the compound of interest. In some embodiments, the purified compound is at least about 75%, 90%, or 99% the compound of interest. In some embodiments, the purified compound is at least about 90%, 91%, 92%, 93%, 94%, 95%, 98%, 99%, or 100% (w/w) the compound of interest. Purity is determined by any known methods, including, without limitation, column chromatography, thin layer chromatography, HPLC analysis, NMR, mass spectrometry, or SDS-PAGE. Purified DNA or RNA is defined as DNA or RNA that is free of exogenous nucleic acids, carbohydrates, and lipids.

In some embodiments, the compound is an intermediate compound. The intermediate compound may be a molecular entity that is formed from preceding intermediates and reacts further to give the final reaction product. In some embodiments, the intermediate compound is a protein precursor, or pro-protein, that is cleaved by an enzyme to produce the mature, functional form of the protein. In some embodiments, the intermediate compound is an inactive enzyme precursor, or zymogen, that requires modification or cleavage to produce the active enzyme.

### XI. Applications

In some aspects, the invention provides methods of treating a patient by introducing an antibody-producing immune cell, modified by passing through a constriction such that a compound that modulates antibody production enters the cell, to the patient. In some embodiments, the cell is isolated from a patient, modified according to the methods disclosed, and introduced back into the patient. For example, a population of B cells is isolated from a patient, passed through the constriction to achieve delivery of a compound that modulates antibody production, and then re-infused into the patient to augment a therapeutic immune response. In some embodiments, the cell is isolated from an individual, modified according to the disclosed methods, and introduced back into the individual. For example, a population of B cells is isolated from an individual, passed through the constriction to achieve delivery of a compound that modulates antibody production, and then re-infused into the patient to enhance antibody production in the individual.

In some embodiments, the invention provides methods of downregulating antibody production in a cell. In some embodiments, delivery of gene-editing compounds such as protein-nucleic acid complexes (e.g., CRISPR) or nucleases (e.g., ZFNs, TALENs), or siRNA-mediated knockdown enables blocking a specific gene involved in pathogenic antibody production. For example, delivery of gene-editing compounds or siRNA can be used to reduce cellular production of auto-reactive antibodies for treating autoimmune disease. In some embodiments, the invention provides methods of controlling antibody production in a cell. For example, delivery of compounds into the cell enables secretion of antibody in response to a stimulus or factor, thereby allowing for positive or negative feedback mechanisms. In some embodiments, delivery of compounds into the cell enables secretion of antibody in a time-dependent manner. For example, the cell produces antibody during a particular time frame or for a particular length of time.

In some embodiments, the invention provides methods of treating a patient by introducing the cell, modified by passing through a constriction such that a compound that induces de novo antibody production enters the cell, to the patient. In some embodiments, the cell is an autologous cell. For example, the cell is isolated from a patient, modified according to the methods disclosed, and introduced back into the patient. In some embodiments, the cell is isolated from an individual, modified according to the disclosed methods, and introduced back into the same individual. In some embodiments, the cell is an allogeneic cell. For example, the cell is isolated from a different individual, modified according to the methods disclosed, and introduced into a patient. In some embodiments, the cell is isolated from an individual, modified according to the disclosed methods, and introduced into a different individual. In some embodiments, a population of cells is isolated from a patient or different individual, passed through the constriction to achieve delivery of a compound that induces de novo antibody production, and then infused into a patient to augment a therapeutic response.

Any of the methods described above are carried out in vitro, ex vivo, or in vivo. For in vivo applications, the device may be implanted in a vascular lumen, e.g., an in-line stent in an artery or vein. In some embodiments, the methods are used as part of a bedside system for ex-vivo treatment of patient cells and immediate reintroduction of the cells into the patient. Such methods could be employed as a means of activating the immune system in response to cancer or infections or in vaccine development. In some embodiments, the method can be implemented in a typical hospital laboratory with a minimally trained technician. In some embodiments, a patient operated treatment system can be used. In some embodiments, the method is implemented using an in-line blood treatment system, in which blood is directly diverted from a patient, passed through the constriction, resulting in compound delivery to blood cells, and directly transfused back into the patient after treatment.

### XII. Systems and Kits

In some aspects, the invention provides a system comprising the constriction, cell suspension, and compound that alters endogenous antibody production in an antibody-producing cell or induces de novo production of antibodies in a cell, for use in the methods disclosed herein. In some embodiments, the endogenous antibody production is enhanced. In some embodiments, the endogenous antibody production is decreased. The system can include any embodiment described for the methods disclosed above, including microfluidic channels or a surface having pores to provide cell-deforming constrictions, cell suspensions, cell perturbations, delivery parameters, compounds to alter or induce antibody production, and/or applications etc. In some embodiment, the cell-deforming constrictions are sized for delivery to antibody-producing cells to alter endogenous antibody production. In some embodiments, the cell-deforming constrictions are sized for delivery of a compound that induces de novo antibody production in a cell. In some embodiments, the delivery parameters, such as operating flow speeds, cell and compound concentration, velocity of the cell in the constriction, and the composition of the cell suspension (e.g., osmolarity, salt concentration, serum content, cell concentration, pH, etc.) are optimized for maximum antibody response of a compound for altering antibody production or inducing de novo antibody production.

Also provided are kits or articles of manufacture for use in delivering a compound to modulate antibody production in a cell. In some embodiments, the kits comprise the compositions described herein (e.g. a microfluidic channel or surface containing pores, cell suspensions, and/or compounds to alter endogenous antibody production or induce de novo antibody production) in suitable packaging. Suitable packaging materials are known in the art, and include, for example, vials (such as sealed vials), vessels, ampules, bottles, jars, flexible packaging (e.g., sealed Mylar or plastic bags), and the like. These articles of manufacture may further be sterilized and/or sealed.

The present disclosure also provides kits comprising components of the methods described herein and may further comprise instruction(s) for performing said methods to alter endogenous antibody production or induce de novo antibody production. The kits described herein may further include other materials, including other buffers, diluents, filters, needles, syringes, and package inserts with instructions for performing any methods described herein; e.g., instructions for modulating antibody production or inducing de novo antibody production.

### XIII. Exemplary Embodiments

1. A method for altering endogenous antibody production in an antibody-producing cell, the method comprising passing a cell suspension comprising the antibody-producing cell through a constriction, wherein said constriction deforms the cell thereby causing a perturbation of the cell such that a compound that alters antibody production enters the antibody-producing cell, wherein endogenous antibody production in said antibody-producing cell is altered.

2. The method of embodiment 1, wherein the endogenous antibody production is enhanced.

3. The method of embodiment 1, wherein the endogenous antibody production is decreased.

4. The method of any one of embodiments 1-3, wherein the constriction is contained within a microfluidic channel.

5. The method of any one of embodiments 1-3, wherein the constriction is a pore or contained within a pore.

6. The method of embodiment 5, wherein the pore is contained in a surface.

7. The method of embodiment 6, wherein the surface is a filter.

8. The method of embodiment 6, wherein the surface is a membrane.

9. The method of any one of embodiments 1-8, wherein the constriction size is a function of the cell diameter.

10. The method of any one of embodiments 1-9, wherein the constriction size is about 20%, about 30%, about 40%, about 50%, about 60%, about 70%, about 80%, about 90%, or about 99% of the cell diameter.

11. The method of any one of embodiments 1-10, wherein the cell suspension comprises a mixed cell population.

12. The method of any one of embodiments 1-11, wherein the cell suspension is whole blood.

13. The method of any one of embodiments 1-10, wherein the cell suspension comprises a purified cell population.

14. The method of any one of embodiments 1-13, wherein the cell suspension comprises mammalian cells.

15. The method of any one of embodiments 1-14, wherein the cell suspension comprises monkey, mouse, dog, cat, horse, rat, sheep, goat, or rabbit cells.

16. The method of any one of embodiments 1-14, wherein the cell suspension comprises human cells.

17. The method of any one of embodiments 1-13, wherein the cell suspension comprises non-mammalian cells.

18. The method of any one of embodiments 1-13 or 17, wherein the cell suspension comprises bacteria, yeast, chicken, frog, insect, or nematode cells.

19. The method of any one of embodiments 1-17, wherein the cell suspension comprises peripheral blood mononuclear cells.

20. The method of any one of embodiments 1-19, wherein the antibody-producing cell is an immune cell.

21. The method of any one of embodiments 1-20, wherein the antibody-producing cell is a B cell or B cell precursor.

22. The method of embodiment 21, wherein the antibody-producing cell is a B cell precursor, naïve B cell, activated B cell, memory B cell, plasma cell, B-1 cell, marginal-zone B cell, follicular B cell, regulatory B cell, or B cell lymphoma cell.

23. The method of embodiment 21 or 22, wherein the antibody-producing cell is a bone-marrow derived B cell precursor.

24. The method of any one of embodiments 1-23, wherein the compound comprises a nucleic acid.

25. The method of embodiment 24, wherein the nucleic acid encodes a siRNA, mRNA, miRNA, lncRNA, tRNA, saRNA or shRNA.

26. The method of embodiment 24, wherein the nucleic acid is a plasmid.

27. The method of any one of embodiments 1-23, wherein the compound comprises a peptide nucleic acid.

28. The method of any one of embodiments 1-23, wherein the compound comprises a protein-nucleic acid complex.

29. The method of embodiment 28, wherein the protein-nucleic acid complex comprises a Cas9 protein and a guide RNA.

30. The method of embodiment 29, further comprising donor DNA.

31. The method of embodiment 24, wherein the nucleic acid encodes a Cas9 protein and a guide RNA.

32. The method of embodiment 31, further comprising donor DNA.

33. The method of any one of embodiments 1-23, wherein the compound comprises a protein or polypeptide.

34. The method of embodiment 33, wherein the protein is a TALEN protein, Zinc finger nuclease, mega nuclease, or CRE recombinase.

35. The method of embodiment 33, wherein the protein is a transcription factor.

36. The method of embodiment 33, wherein the protein is a transposase or integrase enzyme.

37. The method of embodiment 33, wherein the protein is an anti-apoptotic protein.

38. The method of any one of embodiments 1-23, wherein the compound is B-cell activating factor.

39. The method of any one of embodiments 1-23, wherein the compound is a proliferation inducing ligand.

40. The method of any one of embodiments 1-23, wherein the compound is an activator of a B cell receptor signaling molecule.

41. The method of any one of embodiments 1-23, wherein the compound is ATP.

42. The method of any one of embodiments 1-23, wherein the compound is a cell activation factor.

43. The method of any one of embodiments 1-23, wherein the compound is a cell differentiation factor.

44. The method of any one of embodiments 1-23, wherein the compound is a small molecule.

45. The method of any one of embodiments 1-23, wherein the compound is in a nanoparticle.

46. The method of any one of embodiments 1-23, wherein the compound is in a liposome.

47. The method of embodiment 24, wherein the compound is in a virus, viral particle, or vehicle comprising viral capsid.

48. The method of embodiment 24, wherein the compound is in an adeno-associated virus, adeno-associated virus particle, or vehicle comprising adeno-associated virus capsid.

49. The method of any one of embodiments 1-48, wherein in the antibody is a human or humanized antibody.

50. The method of any one of embodiments 1-49, wherein the antibody is an antigen binding antibody variant.

51. The method of any one of embodiments 1-50, wherein the antibody class is IgM, IgG, IgA, IgE, or IgD.

52. The method of any one of embodiments 1-50, wherein the antibody is an antigen binding antibody fragment.

53. The method of embodiment 52, wherein the antibody is a Fab, Fab′, Fab′-SH, Fab2, F(ab′)2, Fv, scFv, scFab, or dsFv fragment.

54. The method of any one of embodiments 1-51, wherein the antibody is a full length antibody.

55. The method of any one of embodiments 1-50, wherein the antibody is a single-domain antibody, nanobody, VHH or VNAR antibody fragment.

56. The method of any one of embodiments 1-50, wherein the antibody is a single-chain antibody.

57. The method of any one of embodiments 1-51, wherein the antibody is a multi-specific antibody.

58. The method of any one of embodiments 1-51, wherein the antibody is an antibody fusion protein.

59. The method of any one of embodiments 1-58, wherein said cell suspension is contacted with the compound before, concurrently, or after passing through the constriction.

60. The method of any one of embodiments 1-2, wherein the channel comprises a constriction length of about 30 μm and a constriction width of about 4 μm.

61. The method of any one of embodiments 1-4, wherein the channel comprises a constriction length of about 10 μm and a constriction width of about 4 μm.

62. The method of any one of embodiments 1-3 or 5, wherein the pore size is about 0.4 μm, about 4 μm, about 5 μm, about 8 μm, about 10 μm, about 12 μm, or about 14 μm.

63. The method of any one of embodiments 1-62, wherein the method is performed between about −5° C. and about 45° C.

64. The method of any one of embodiment 1-63, wherein the endogenous antibody production is altered by at least about 25%, about 50%, about 75%, about 100%, about 150%, about 200%, or more than about 200%.

65. The method of any one of embodiment 1-63, wherein the endogenous antibody production is sustained for about 25%, about 50%, about 75%, about 100%, about 150%, about 200%, or more than about 200% longer than antibody production by cells that did not pass through the constriction.

66. A method of treating a patient by introducing the antibody-producing immune cell modified according to any one of embodiments 1-16 or 19-65 to the patient.

67. The method of embodiment 66, wherein the cell is isolated from a patient, modified according to the methods of any one of embodiments 1-16 or 19-65, and introduced back into the patient.

68. The method of embodiment 66, wherein the cell is isolated from a different individual, modified according to the methods of any one of embodiments 1-16 or 19-65, and introduced into a patient.

69. A method of enhancing antibody production in an individual by introducing the antibody-producing immune cell modified according to any one of embodiments 1-16 or 19-65 to the individual.

70. The method of embodiment 69, wherein the cell is isolated from an individual, modified according to the methods of any one of embodiments 1-16 or 19-65, and introduced back into the individual.

71. The method of embodiment 69, wherein the cell is isolated from an individual, modified according to the methods of any one of embodiments 1-16 or 19-65, and introduced into a different individual.

72. The method of any one of embodiments 1-71, wherein the method further comprises the step of contacting the cell with an electric field generated by at least one electrode.

73. A system comprising the constriction, cell suspension, and compound for use in the methods of any one of embodiments 1-72.

74. The system of embodiment 73, further comprising at least one electrode to generate an electric field.

75. A method for inducing de novo antibody production in a cell, the method comprising passing a cell suspension through a constriction, wherein said constriction deforms the cell thereby causing a perturbation of the cell such that a compound that initiates antibody productions enters the cell, wherein de novo antibody production in said cell is induced.

76. The method of embodiment 75, wherein the constriction is contained within a microfluidic channel.

77. The method of embodiment 75, wherein the constriction is a pore or contained within a pore.

78. The method of embodiment 77, wherein the pore is contained in a surface.

79. The method of embodiment 78, wherein the surface is a filter.

80. The method of embodiment 78, wherein the surface is a membrane.

81. The method of any one of embodiments 75-80, wherein the constriction size is a function of the cell diameter.

82. The method of any one of embodiments 75-81, wherein the constriction size is about 20%, about 30%, about 40%, about 50%, about 60%, about 70%, about 80%, about 90%, or about 99% of the cell diameter.

83. The method of any one of embodiments 75-82, wherein the cell suspension comprises a mixed cell population.

84. The method of any one of embodiments 75-83 wherein the cell suspension is whole blood.

85. The method of any one of embodiments 75-84, wherein the cell suspension comprises a purified cell population.

86. The method of any one of embodiments 75-85, wherein the cell suspension comprises mammalian cells.

87. The method of any one of embodiments 75-86, wherein the cell suspension comprises monkey, mouse, dog, cat, horse, rat, sheep, goat, or rabbit cells.

88. The method of any one of embodiments 75-86, wherein the cell suspension comprises human cells.

89. The method of any one of embodiments 75-85, wherein the cell suspension comprises non-mammalian cells.

90. The method of any one of embodiments 75-85 or 89, wherein the cell suspension comprises bacteria, yeast, chicken, frog, insect, or nematode cells.

91. The method of any one of embodiments 75-89, wherein the cell suspension comprises peripheral blood mononuclear cells.

92. The method of any one of embodiments 75-91, wherein the cell is an immune cell, stem cell, bone marrow-derived progenitor cell, erythrocyte precursor, fibroblast, cardiac cell, or cell line cell.

93. The method of any one of embodiment 75-92, wherein the cell is a B cell, T cell, monocyte, macrophage, neutrophil, eosinophil, dendritic cell, basophil, NK cell, NKT cell, mast cell, or stem cell.

94. The method of any one of embodiments 75-93, wherein the cell is a B cell or B-cell precursor.

95. The method of embodiment 94, wherein the cell is a B cell precursor, naïve B cell, activated B cell, memory B cell, plasma cell, B-1 cell, marginal-zone B cell, follicular B cell, regulatory B cell, or B cell lymphoma cell.

96. The method of embodiments 94 or 95, wherein the cell is a bone-marrow derived B cell precursor.

97. The method of any one of embodiments 75-96, wherein the compound comprises a nucleic acid.

98. The method of embodiment 97, wherein the nucleic acid encodes an immunoglobulin.

99. The method of embodiment 98, wherein the nucleic acid is integrated into the cell genome.

100. The method of embodiment 98, wherein the nucleic acid is not integrated into the cell genome.

101. The method of embodiment 97, wherein the nucleic acid encodes a siRNA, mRNA, miRNA, lncRNA, tRNA, saRNA or shRNA.

102. The method of embodiment 97, wherein the nucleic acid is a plasmid.

103. The method of any one of embodiments 75-96, wherein the compound comprises a peptide nucleic acid.

104. The method of any one of embodiments 75-96, wherein the compound comprises a protein-nucleic acid complex.

105. The method of embodiment 104, wherein the protein-nucleic acid complex comprises a Cas9 protein and a guide RNA.

106. The method of embodiment 105, further comprising donor DNA.

107. The method of embodiment 97, wherein the nucleic acid encodes a Cas9 protein and a guide RNA.

108. The method of embodiment 107, further comprising donor DNA.

109. The method of any one of embodiments 75-96, wherein the compound comprises a protein or polypeptide.

110. The method of embodiment 109, wherein the protein is a TALEN protein, Zinc finger nuclease, mega nuclease, or CRE recombinase.

111. The method of embodiment 109, wherein the protein is a transcription factor.

112. The method of embodiment 109, wherein the protein is a transposase or integrase enzyme.

113. The method of any one of embodiments 75-96, wherein the compound is B-cell activating factor.

114. The method of any one of embodiments 75-96, wherein the compound is a proliferation inducing ligand.

115. The method of any one of embodiments 75-96, wherein the compound is an activator of a B cell receptor signaling molecule.

116. The method of any one of embodiments 75-96, wherein the compound is a cell activation factor.

117. The method of any one of embodiments 75-96, wherein the compound is a cell differentiation factor.

118. The method of any one of embodiments 75-96, wherein the compound is a small molecule.

119. The method of any one of embodiments 75-96, wherein the compound is in a nanoparticle.

120. The method of any one of embodiments 75-96, wherein the compound is in a liposome.

121. The method of embodiment 97, wherein the compound is in a virus, viral particle, or vehicle comprising viral capsid.

122. The method of embodiment 97, wherein the compound is in an adeno-associated virus, adeno-associated virus particle, or vehicle comprising adeno-associated virus capsid.

123. The method of any one of embodiments 75-122, wherein in antibody is a human or humanized antibody.

124. The method of any one of embodiments 75-123, wherein the antibody is an antigen binding antibody variant.

125. The method of any one of embodiments 75-124, wherein the antibody class is IgM, IgG, IgA, IgE, or IgD.

126. The method of any one of embodiments 75-124, wherein the antibody is an antigen binding antibody fragment.

127. The method of embodiment 126, wherein the antibody is a Fab, Fab′, Fab′-SH, Fab2, F(ab′)2, Fv, scFv, scFab, or dsFv fragment.

128. The method of any one of embodiments 75-125, wherein the antibody is a full length antibody.

129. The method of any one of embodiments 75-124, wherein the antibody is a single-domain antibody, nanobody, VHH or VNAR antibody fragment.

130. The method of any one of embodiments 75-124, wherein the antibody is a single-chain antibody.

131. The method of any one of embodiments 75-125, wherein the antibody is a multi-specific antibody.

132. The method of any one of embodiments 75-125, wherein the antibody is an antibody fusion protein.

133. The method of any one of embodiments 75-132, wherein said cell suspension is contacted with the compound before, concurrently, or after passing through the constriction.

134. The method of any one of embodiments 75-76, wherein the channel comprises a constriction length of about 30 μm and a constriction width of about 4 μm.

135. The method of any one of embodiments 75-76, wherein the channel comprises a constriction length of about 10 μm and a constriction width of about 4 μm.

136. The method of any one of embodiments 75 or 77, wherein the pore size is about 0.4 μm, about 4 μm, about 5 μm, about 8 μm, about 10 μm, about 12 μm, or about 14 μm.

137. The method of any one of embodiments 75-136, wherein the method is performed between about −5° C. and about 45° C.

138. A method of treating a patient by introducing the cell modified according to any one of embodiments 75-88 or 91-137 to the patient.

139. The method of embodiment 138, wherein the cell is isolated from a patient, modified according to the methods of any one of embodiments 75-88 or 91-137, and introduced back into the patient.

140. The method of embodiment 138, wherein the cell is isolated from a different individual, modified according to the methods of any one of embodiments 75-88 or 91-137, and introduced into a patient.

141. A method of inducing de novo antibody production in an individual by introducing the cell modified according to any one of embodiments 75-88 or 91-137 to the individual.

142. The method of embodiment 141, wherein the cell is isolated from an individual, modified according to the methods of any one of embodiments 75-88 or 91-137, and introduced back into the individual.

143. The method of embodiment 141, wherein the cell is isolated from an individual, modified according to the methods of any one of embodiments 75-88 or 91-137, and introduced into a different individual.

144. The method of any one of embodiments 75-143, wherein the method further comprises the step of contacting the cell with an electric field generated by at least one electrode.

145. A system comprising the constriction, cell suspension, and compound for use in the methods of any one of embodiments 75-144.

146. The system of embodiment 145, further comprising at least one electrode to generate an electric field.

### XIV. Examples

The following examples are given for the purpose of illustrating various embodiments of the disclosure and are not meant to limit the present disclosure in any fashion. One skilled in the art will appreciate readily that the present disclosure is well adapted to carry out the objects and obtain the ends and advantages mentioned, as well as those objects, ends and advantages inherent herein. Changes therein and other uses which are encompassed within the spirit of the disclosure as defined by the scope of the claims will occur to those skilled in the art.

### Example 1: Constriction-Mediated Delivery of mRNA Encoding an Antibody to Induce De Novo B Cell Antibody Production

Spleens are harvested from C57BL/6J female mice and mashed through a 70 μm cell strainer. Red blood cells are lysed and the B cells are further isolated from the cell suspension using a B cell isolation kit (Miltenyi Biotec) as per the instructions. B cells are suspended in PBS at a concentration of 1-10×106 cells per mL with mRNA at a concentration of 0.1-0.4 mg/mL. As a control, B cells are processed without mRNA. The cell suspensions are processed through either 10-4 chip or 30-4 chip at a pressure of 90 psi. Post processing, the cells are washed three times in R10 with centrifugation in between and finally resuspended in R10 and plated at 10-50,000 cells/well in a 96 well u-bottom, with R848 1 ug/ml+IL-2 100 units/ml. The cells are cultured for 3 days after which the supernatants are collected and assayed for total antibody content by ELISA. Total antibody content produced by B cells to which mRNA is delivered is compared to total antibody produced by B cells that do not receive mRNA.

### Example 2: Constriction-Mediated Delivery of a Compound to Enhance B Cell Antibody Production

Spleens are harvested from C57BL/6J female mice and mashed through a 70 μm cell strainer. Red blood cells are lysed and the B cells are further isolated from the cell suspension using a B cell isolation kit (Miltenyi Biotec) as per the instructions. B cells are suspended in PBS at a concentration of 1-10×10⁶ cells per mL with the compound to enhance B cell antibody production. As a control, B cells are processed without the compound. The cell suspensions are processed through either 10-4 chip or 30-4 chip at a pressure of 90 psi. Post processing, the cells are washed three times in R10 with centrifugation in between and finally resuspended in R10 and plated at 10-50,000 cells/well in a 96 well u-bottom, with R848 1 ug/ml+IL-2 100 units/ml. The cells are cultured for 3 days after which the supernatants are collected and assayed for total antibody content by ELISA. Total antibody content produced by B cells to which the compound is delivered is compared to total antibody produced by B cells that do not receive the compound.

1. A method for altering endogenous antibody production in an antibody-producing cell, the method comprising passing a cell suspension comprising the antibody-producing cell through a constriction, wherein said constriction deforms the cell thereby causing a perturbation of the cell such that a compound that alters antibody production enters the antibody-producing cell, wherein endogenous antibody production in said antibody-producing cell is altered.

2. The method of claim 1, wherein the endogenous antibody production is enhanced.

3. The method of claim 1, wherein the endogenous antibody production is decreased.

4. The method of claim 1, wherein:
(a) the constriction is contained within a microfluidic channel; or
(b) wherein the constriction is a pore or contained within a pore.

5. (canceled)

6. The method of claim 4, wherein the pore is contained in a surface.

7. The method of claim 6, wherein the surface is a filter or a membrane.

8. (canceled)

9. The method of claim 1, wherein the constriction size is a function of the cell diameter.

10. The method of claim 1, wherein the constriction size is about 20%, about 30%, about 40%, about 50%, about 60%, about 70%, about 80%, about 90%, or about 99% of the cell diameter.

11. The method of claim 1, wherein the cell suspension:
(a) comprises a mixed cell population and/or is whole blood; or
(b) comprises a purified cell population.

12-13. (canceled)

14. The method of claim 1, wherein the cell suspension comprises mammalian cells.

15. (canceled)

16. The method of claim 1, wherein the cell suspension comprises human cells.

17-18. (canceled)

19. The method of claim 1, wherein:
(a) the cell suspension comprises peripheral blood mononuclear cells; and/or
(b) the antibody-producing cell is an immune cell.

20. (canceled)

21. The method of claim 1, wherein the antibody-producing cell is a B cell or B cell precursor.

22. The method of claim 21, wherein the antibody-producing cell is a bone-marrow derived B cell precursor, naive B cell, activated B cell, memory B cell, plasma cell, B-1 cell, marginal-zone B cell, follicular B cell, regulatory B cell, or B cell lymphoma cell.

23. (canceled)

24. The method of claim 1, wherein the compound comprises:
(a) a nucleic acid;
(b) a peptide nucleic acid;
(c) a protein-nucleic acid complex;
(d) a protein or polypeptide;
(e) a B-cell activating factor
(f) a proliferation inducing ligand,
(g) a B cell receptor signaling molecule;
(h) ATP;
(i) a cell activation factor;
(j) a cell differentiation factor;
(k) a small molecule;
(l) a nanoparticle; and/or
(m) a liposome.

25. The method of claim 24, wherein:
(a) the compound comprises a nucleic acid; wherein:
(i) the nucleic acid encodes a siRNA, mRNA, miRNA, lncRNA, tRNA, saRNA or shRNA;
(ii) the nucleic acid is a plasmid;
(iii) the nucleic acid encodes a Cas9 protein and a guide RNA; or
(iv) the compound is in a virus, viral particle, or vehicle comprising viral capsid;
(b) the compound comprises a protein-nucleic acid complex and the protein-nucleic acid complex comprises a Cas9 protein and a guide RNA; or
(c) the compound comprises a protein or polypeptide and wherein the protein is:
(i) a TALEN protein, Zinc finger nuclease, mega nuclease, or CRE recombinase;
(ii) a transcription factor;
(iii) a transposase or integrase enzyme; or
(iv) an anti-apoptotic protein.

26-29. (canceled)

30. The method of claim 24, wherein:
(a) the compound comprises a protein-nucleic acid complex, wherein the protein-nucleic acid complex comprises a Cas9 protein and a guide RNA, and wherein the compound further comprising donor DNA;
(b) the compound comprises a nucleic acid; wherein the nucleic acid encodes a Cas9 protein and a guide RNA, and wherein the compound further comprises donor DNA; or
(c) the compound comprises a nucleic acid, wherein the compound is in a virus, viral particle, or vehicle comprising viral capsid, and wherein the virus is adeno-associated virus.

31-48. (canceled)

49. The method of claim 1, wherein in the antibody is a human or humanized antibody.

50. The method of claim 1, wherein: the antibody is an antigen binding antibody variant; and/or the antibody class is IgM, IgG, IgA, IgE, or IgD.

51. (canceled)

52. The method of claim 1, wherein:
(a) the antibody is an antigen binding antibody fragment;
(b) the antibody is a full length antibody;
(c) the antibody is a single-domain antibody, nanobody, V_{H}H or V_{NA}R antibody fragment;
(d) the antibody is a single-chain antibody;
(e) the antibody is a multi-specific antibody; or
(f) the antibody is an antibody fusion protein.

53. The method of claim 52, wherein the antibody is an antigen binding antibody fragment, wherein the antibody is a Fab, Fab′, Fab′-SH, Fab₂, F(ab′)₂, Fv, scFv, scFab, or dsFv fragment.

54-58. (canceled)

59. The method of claim 1, wherein said cell suspension is contacted with the compound before, concurrently, or after passing through the constriction.

60. The method of claim 4, wherein:
(a) the channel comprises a constriction length of about 30 pm and a constriction width of about 4 pm;
(b) the channel comprises a constriction length of about 10 pm and a constriction width of about 4 pm; or
(c) the pore size is about 0.4 pm, about 4 pm, about 5 pm, about 8 pm, about 10 pm, about 12 pm, or about 14 pm.

61-64. (canceled)

65. The method of claim 1, wherein the endogenous antibody production is sustained for about 25%, about 50%, about 75%, about 100%, about 150%, about 200%, or more than about 200% longer than antibody production by cells that did not pass through the constriction.

66. A method of treating a patient and/or enhancing antibody production in a patient by introducing the antibody-producing immune cell modified according to claim 1 to the patient.

67. A method of treating a patient and/or enhancing antibody production in a patient, wherein the cell is isolated from the patient or from a different individual, modified according to method of claim 1, and introduced into the patient.

68-71. (canceled)

72. The method of claim 1, wherein the method further comprises the step of contacting the cell with an electric field generated by at least one electrode.

73. A system comprising the constriction, cell suspension, and compound for use in the methods of claim 1.

74. (canceled)

75. A method for inducing de novo antibody production in a cell, the method comprising passing a cell suspension through a constriction, wherein said constriction deforms the cell thereby causing a perturbation of the cell such that a compound that initiates antibody productions enters the cell, wherein de novo antibody production in said cell is induced.

76-137. (canceled)

138. A method of treating a patient and/or inducing de novo antibody production in a patient by introducing the cell modified according to claim 75 to the patient.

139. A method of treating a patient and/or inducing de novo antibody production in a patient, wherein the cell is isolated from a patient or from a different individual, modified according to the methods of claim 75, and introduced back into the patient.

140-144. (canceled)

145. A system comprising the constriction, cell suspension, and compound for use in the methods of claim 75.

146. (canceled)
